(19) United States
(12) Patent Application Publication
(10) Pub. No.: US 2024/0251758 A1
(43) Pub. Date: Aug. 1, 2024

(54) ANIMAL MONITORING SYSTEM, ANIMAL MONITORING SERVER, ANIMAL MONITORING METHOD, ANIMAL MONITORING PROGRAM, AND RECTAL TEMPERATURE ESTIMATION MODEL

(71) Applicant: PetVoice Co., Ltd., Tokyo (JP)

(72) Inventors: Atsushi FUKADA, Tokyo (JP); Keigo OSHIRO, Tokyo (JP)

(73) Assignee: PetVoice Co., Ltd., Tokyo (JP)

(21) Appl. No.: 17/927,632

(22) PCT Filed: Jun. 28, 2022
(86) PCT No.: PCT/JP2022/025678

§ 371 Date: Nov. 23, 2022

(30) Foreign Application Priority Data
Jun. 30, 2021 (JP) 2021-109196
Sep. 9, 2021 (JP) 2021-146688

(51) Int. Cl. A01K 29/00; A01K 27/00; G01K 13/20

(52) U.S. Cl. CPC A01K 29/005; G01K 13/20; A01K 27/009

(57) ABSTRACT

Provided are an animal monitoring system, an animal monitoring server, an animal monitoring method, an animal monitoring program, and a rectal temperature estimation model capable of accurately estimating a rectal temperature of a monitored animal such as a pet without causing the animal to feel annoying. An animal monitoring system 1 capable of estimating a rectal temperature of a monitored animal 6 includes: a body surface temperature detection unit 500 attached to the monitored animal 6 and configured to detect a body surface temperature of the monitored animal 6; and a rectal temperature estimation unit 210 configured to estimate the rectal temperature of the monitored animal by applying the body surface temperature detected by the body surface temperature detection unit 500 to a rectal temperature estimation model that receives, as rectal temperature training data, a combination of the body surface temperature of the monitored animal 6 and the rectal temperature of the monitored animal 6, and outputs the rectal temperature of the monitored animal 6 from the body surface temperature of the monitored animal 6 by machine learning using the rectal temperature training data.

### TECHNICAL FIELD

The present invention relates to an animal monitoring system, an animal monitoring server, an animal monitoring method, an animal monitoring program, and a rectal temperature estimation model. More particularly, the present invention relates to an animal monitoring system, an animal monitoring server, an animal monitoring method, an animal monitoring program, and a rectal temperature estimation model capable of estimating a rectal temperature of a monitored animal such as a pet.

### BACKGROUND ART

In recent years, the importance of daily health care of pets has been widely recognized among their owners. A pet body temperature is an important indicator for grasping a pet health condition. In most cases, a rectal temperature is measured in the body temperature measurement of animals including pets. For example, the rectal temperature of a dog is measured by inserting a probe thermometer including a thermistor into the rectum of the dog. However, it is difficult to measure the rectal temperature of an animal in ordinary homes. Therefore, there is a demand for measuring the rectal temperature of an animal by a simple method instead of the direct measurement of the rectal temperature.

Conventionally, there has been known a body surface temperature measurement device that measures a body surface temperature of an animal, the body surface temperature measurement device including: a temperature sensing unit that includes an infrared ray detection sensor and a cover that covers the infrared ray detection sensor; and a belt that causes the temperature sensing unit to closely contact a body surface of the animal or that fixes the temperature sensing unit to a collar to be mounted on the animal or a body belt to be mounted on the animal, in which in a case where the body surface temperature measurement device is mounted on the animal, the cover is pressed against the body surface of the animal, and at least a part of a contact surface of the cover, which contacts the body surface of the animal, is formed into a curved surface that bulges toward a body surface side of the animal (For example, see Patent Literature 1).

### CITATION LIST

### Patent Literature

Patent Literature 1: WO 2018/105471 A

### SUMMARY OF INVENTION

### Technical Problem

However, in the body surface temperature measurement device described in Patent Literature 1, since it is necessary to provide a dome-shaped cover covering the infrared ray sensor, the measurement device becomes large in size, and causes the animal to feel annoying while wearing the measurement device. Therefore, the body surface temperature measurement device is easily damaged by the animal attempting to remove the body surface temperature measurement device by itself or the like. In addition, even in a case where the infrared ray sensor is provided with the cover as in the body surface temperature measurement device described in Patent Literature 1, it is not always possible to obtain an accurate body temperature detection result for, for example, an animal having a large amount of hair.

Therefore, an object of the present invention is to provide an animal monitoring system, an animal monitoring server, an animal monitoring method, an animal monitoring program, and a rectal temperature estimation model capable of accurately estimating a rectal temperature of an animal such as a pet without causing the animal to feel annoying.

### Solution to Problem

In order to achieve the above object, the present invention provides an animal monitoring system capable of estimating a rectal temperature of a monitored animal, the animal monitoring system including: a body surface temperature detection unit attached to the monitored animal and configured to detect a body surface temperature of the monitored animal; and a rectal temperature estimation unit configured to estimate the rectal temperature of the monitored animal by applying the body surface temperature detected by the body surface temperature detection unit to a rectal temperature estimation model that receives, as rectal temperature training data, a combination of the body surface temperature of the monitored animal and the rectal temperature of the monitored animal, and outputs the rectal temperature of the monitored animal from the body surface temperature of the monitored animal by machine learning using the rectal temperature training data.

In addition, in order to achieve the above object, the present invention provides a rectal temperature estimation model that causes a processor to function to output a rectal temperature of a monitored animal when a body surface temperature of the monitored animal is input, in which the rectal temperature estimation model is learned using, as training data, a combination of the body surface temperature of the monitored animal and the rectal temperature of the monitored animal, and in the learning, a relationship between the body surface temperature and the rectal temperature is learned using the training data to estimate the rectal temperature of the monitored animal.

### Advantageous Effects of Invention

In accordance with the animal monitoring system, the animal monitoring server, the animal monitoring method, the animal monitoring program, and the rectal temperature estimation model according to the present invention, it is possible to provide the animal monitoring system, the animal monitoring server, the animal monitoring method, the animal monitoring program, and the rectal temperature estimation model capable of accurately estimating the rectal temperature of the monitored animal such as a pet without causing the animal to feel annoying.

### BRIEF DESCRIPTION OF DRAWINGS

FIG. 1 is a schematic view of an animal monitoring system according to the present embodiment.

FIG. 2 is a schematic view of an appearance of a monitoring device according to the present embodiment.

FIG. 3 is a schematic view of an appearance of a relay device according to the present embodiment.

FIG. 4 is a functional configuration block diagram of a server according to the present embodiment.

FIG. 5 is a functional configuration block diagram of the relay device according to the present embodiment.

FIG. 6 is a functional configuration block diagram of the monitoring device and an information terminal according to the present embodiment.

FIG. 7 is a flowchart of user registration and device linking processing.

FIG. 8 is a drawing of output contents in the information terminal at the time of user registration.

FIG. 9 is a flowchart of user registration and device linking processing.

FIG. 10 is a drawing of output contents of information regarding the linked relay device in the information terminal.

FIG. 11 is a flowchart of processing when a monitored animal and a feature amount are registered in the server.

FIG. 12 is a drawing of output contents in the information terminal when the monitored animal is registered.

FIG. 13 is a flowchart of estimation processing of a rectal temperature and a behavior of the monitored animal.

FIG. 14 is a drawing illustrating a state in which an output unit of the information terminal displays the rectal temperature and the like.

FIG. 15 is a flowchart of power saving control processing in the monitoring device.

FIG. 16 is a flowchart of another power saving control processing in the monitoring device.

### DESCRIPTION OF EMBODIMENT

### Embodiment

### <Outline of Animal Monitoring System 1>

An animal monitoring system 1 according to the present embodiment is a system that estimates a rectal temperature and/or a behavior of an animal wearing a monitoring device on the basis of a state or a motion of the animal, and enables an information terminal of a user to refer to an estimation result. That is, the animal monitoring system 1 detects a body surface temperature and/or a motion of the animal by the monitoring device attached to the animal, and estimates the rectal temperature of the animal on the basis of the detected body surface temperature, the detected motion, and/or a feature amount related to a characteristic such as a hair amount of the animal. In addition, the animal monitoring system 1 can estimate the behavior of the animal on the basis of the detected body surface temperature, the detected motion, and/or the feature amount of the animal. Furthermore, the animal monitoring system 1 can reduce power consumption of the monitoring device on the basis of the rectal temperature and/or the behavior estimated for the animal as a monitoring target (hereinafter, the animal may be referred to as a “monitored animal”).

In the present embodiment, the “motion” refers to a movement, a posture, a direction, and the like of the monitored animal that can be measured by a measurement device such as a sensor, and the “behavior” refers to a target reaction (e.g., exercise, eating, sleeping, grooming, resting, and the like) of the monitored animal.

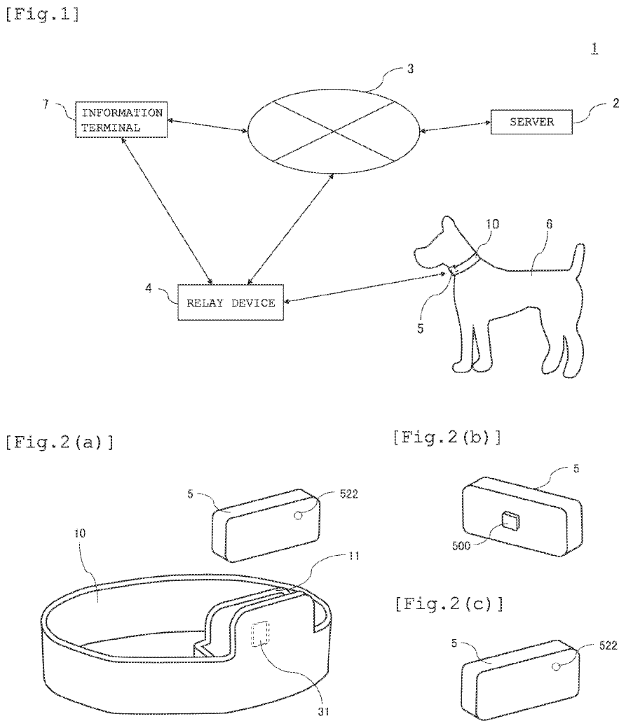

FIG. 1 illustrates an outline of an example of the animal monitoring system according to the present embodiment. Note that FIG. 1 illustrates an example in which each of the number of servers 2, the number of relay devices 4 (home devices), the number of monitoring devices 5 (collar-type devices), and the number of information terminals 7 is one. However, the animal monitoring system 1 may include a plurality of servers 2, relay devices 4, monitoring devices 5, and/or information terminals 7. Hereinafter, a case where the number of respective elements is one in principle will be described as an example for the simplicity of explanation.

Specifically, the animal monitoring system 1 includes the server 2 that executes estimation and the like of a rectal temperature and/or a behavior of a monitored animal 6, the monitoring device 5 that is fixed to a belt 10 or the like attached to the monitored animal 6 and detects a body surface temperature and/or a motion of the monitored animal 6, and the information terminal 7 that can refer to an estimation result by the server 2. In the animal monitoring system 1, the monitoring device 5 is provided so as to be able to bidirectionally communicate with the server 2 via the relay device 4. The server 2, the relay device 4, and the information terminal 7 are connected so as to be able to bidirectionally communicate with each other via a communication network 3. In addition, the monitoring device 5 and the relay device 4 and/or the information terminal 7 and the relay device 4 can be connected so as to be able to bidirectionally communicate with each other by short-range wireless communication (for example, Bluetooth (registered trademark) or the like). Although the relay device 4 is directly connected to the communication network 3 in the example of FIG. 1, the relay device 4 may be connected to the communication network 3 via a router (not illustrated) or a mobile phone network between the communication network 3 and the relay device 4. In addition, the monitoring device 5 may be directly connected to the server 2 via the communication network 3 without via the relay device 4.

Here, there is a predetermined correlation between the rectal temperature and the body surface temperature of the monitored animal. However, the correlation varies depending on the feature amount of the monitored animal (e.g., a kind, a size, a hair amount, and the like of the monitored animal). Therefore, the server 2 generates, by machine learning, a rectal temperature estimation model that outputs the rectal temperature of the monitored animal by applying the body surface temperature of the monitored animal and/or the feature amount of the monitored animal thereto, and estimates the rectal temperature using the generated rectal temperature estimation model. That is, the monitoring device 5 detects the body surface temperature and/or the motion of the monitored animal 6, and supplies information regarding the detected body surface temperature and/or motion to the server 2 via the relay device 4 and the communication network 3. The server 2 estimates and specifies the rectal temperature and/or the behavior (for example, a behavior indicating that the monitored animal 6 is eating, sleeping, resting, defecating, or the like) of the monitored animal 6 on the basis of the information received from the monitoring device 5. The server 2 stores the estimation result. A user uses the information terminal 7 to request the estimation result from the server 2 via the communication network 3 for the purpose of grasping a state of the monitored animal 6. The server 2 supplies the estimation result to the information terminal 7 via the communication network 3 in response to the request. As a result, even when the user is located at a position physically separated from the monitored animal 6, the user can grasp the estimated rectal temperature and/or behavior of the monitored animal 6 by causing the information terminal 7 to output the estimated rectal temperature and/or behavior (for example, to an output unit such as a display unit).

In addition, the state of the monitored animal 6 can also be grasped using the relay device 4. That is, the user can request the estimation result from the server 2 using the relay device 4. The server 2 supplies the estimation result to the relay device 4 via the communication network 3 in response to the request. As a result, even in a case where the user does not have the information terminal 7, the user can grasp the estimated rectal temperature and/or behavior of the monitored animal 6 by the relay device 4.

Note that the relay device 4 is installed in an area where the monitored animal 6 is present (for example, in a case where the monitored animal is a pet reared in a house, the relay device 4 is in an area in the house and an outdoor area within a predetermined range from the house). One or more of the relay devices 4 may be installed in the area. Even when the plurality of relay devices 4 are installed in the area, each of the plurality of relay devices 4 is connected to the server 2 via the communication network 3 so as to be able to bidirectionally communicate therewith. In this case, the plurality of relay devices 4 may be connected to the communication network 3 via a router, and the connection may be a wired LAN or wireless LAN connection.

### <Outline of Appearance of Monitoring Device 5>

FIG. 2 illustrates an outline of an example of an appearance of the monitoring device according to the present embodiment. Specifically, FIG. 2(a) illustrates an outline of the monitoring device 5 and the belt 10 to be attached to the monitored animal 6, FIG. 2(b) illustrates a back surface of the monitoring device 5, and FIG. 2(c) illustrates a front surface of the monitoring device 5.

As described later, the monitoring device 5 includes a body surface temperature detection unit 500 that detects the body surface temperature of the monitored animal 6 and/or a motion detection unit that detects the motion of the monitored animal 6. The monitoring device 5 is fixed to the belt 10 to be attached to, for example, the neck or the like of the monitored animal 6. The form of the monitoring device 5 is not particularly limited as long as the monitoring device 5 has a shape and a size that do not cause the monitored animal 6 to feel annoying. As an example, the monitoring device 5 can be configured as a thin and small rectangular parallelepiped electronic device. For example, by providing a device mounting portion 11 in which the monitoring device 5 can be mounted on the inner side of the belt 10 as a collar to be attached to the neck of the monitored animal 6, and by mounting the monitoring device 5 on the device mounting portion 11, it is possible to fix the monitoring device 5 to the belt 10. While a method for mounting the monitoring device 5 on the device mounting portion 11 is not limited, for example, the monitoring device 5 can slide into and be mounted on the device mounting portion 11.

Here, as illustrated in FIG. 2(b), the monitoring device 5 includes on the back surface thereof the body surface temperature detection unit 500 protruding to the outside. As illustrated in FIG. 2(a), the device mounting portion 11 includes a hole portion 31 on the body surface side of the monitored animal 6. When the monitoring device 5 is mounted on the device mounting portion 11, the body surface temperature detection unit 500 is exposed from the device mounting portion 11 while facing the body surface of the monitored animal 6 through the hole portion 31. In addition, as illustrated in FIG. 2(c), the monitoring device 5 may include a monitoring device output unit 522 that outputs a motion status and the like of the monitoring device 5 on the front surface. Note that a snap button or other fall prevention portions that prevent the monitoring device 5 from falling off the belt 10 may be provided on the belt 10 (not illustrated).

### <Outline of Appearance of Relay Device 4>

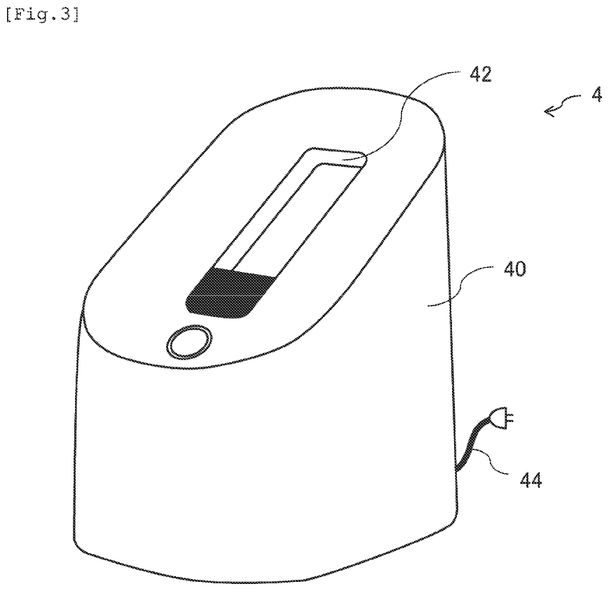

FIG. 3 illustrates an outline of an example of an appearance of the relay device according to the present embodiment.

The shape of the relay device 4 is not particularly limited as long as the relay device 4 is less likely to roll over or the like in the area where the monitored animal 6 is present. For example, the relay device 4 may have a columnar shape having an elliptical cross section, the columnar shape being obtained by obliquely cutting its upper portion from the viewpoint of making the upper portion easily viewable from the user. As an example, the relay device 4 includes a main body portion 40 and a mounting portion 42 that is provided on an upper surface of the main body portion 40 and in which the monitoring device 5 can be fitted and mounted. The mounting portion 42 includes a terminal (not illustrated) that electrically connects the relay device 4 and the monitoring device 5. As a result, it is possible to mount the monitoring device 5 on the mounting portion 42 of the relay device 4 and directly connect the monitoring device 5 to the relay device 4. For example, it is possible to easily execute update of information stored in the monitoring device 5, update of a program, and the like via the relay device 4 regardless of the remaining battery capacity of the monitoring device 5. Note that the relay device 4 receives power supply from the outside through an insertion plug 44 (Note that the insertion plug 44 may be configured using a USB cable, and the relay device 4 may receive power supply by inserting the USB cable into a predetermined USB port of, for example, a mobile communication terminal such as a smartphone, other electronic devices, or an external power source). In addition, the relay device 4 may include an output unit (not illustrated) that outputs predetermined information at any position on the surface of the main body portion 40.

### <About Monitored Animal>

The monitored animal is not particularly limited as long as the rectal temperature of the animal can be measured. Examples of the monitored animal include animals such as companion animals (e.g., pets such as a dog, a cat, a rabbit, a ferret, a hamster, a monkey, and the like), rearing animals (e.g., a tiger, a lion, and the like reared in a zoo and the like), and/or livestock (e.g., a horse, cattle, a pig, a sheep, a goat, and the like).

Note that the information terminal 7 is, for example, a mobile communication terminal, a smartphone, a notebook computer, and/or a tablet PC. Furthermore, the communication network 3 is a mobile phone network and/or a communication network such as the Internet. In addition, the server 2 may include a plurality of servers. That is, functions of the server 2 may be distributed to the plurality of servers. In addition, some or all of the functions of the server 2 may be included in the relay device 4 and/or the monitoring device 5. The communication network 3 may also include a communication network such as a wired LAN and a wireless LAN. In the following, the animal monitoring system 1 according to the present embodiment will be described in detail. It should be noted that names, numerical values, quantities, and the like in the above description and the following description are merely examples, and the present invention is not limited to these names, numerical values, quantities, and the like.

### <Details of Animal Monitoring System 1>

The animal monitoring system 1 is a system capable of estimating the rectal temperature of the monitored animal 6, and includes constituent elements such as the server 2, the relay device 4, the monitoring device 5, and the information terminal 7. Hereinafter, the details of each constituent element will be described. In the following description, it should be noted that a case where predetermined information is supplied or acquired from one member to another member does not preclude a configuration in which the predetermined information passes through still another member or the communication network 3 between the one member and the another member. In addition, one or more constituent members included in one constituent element may be included in another constituent element. Furthermore, in the following description, a constituent member referred to as an “estimation unit” may be a prediction unit that predicts contents estimated by each constituent member.

### [Details of Server 2]

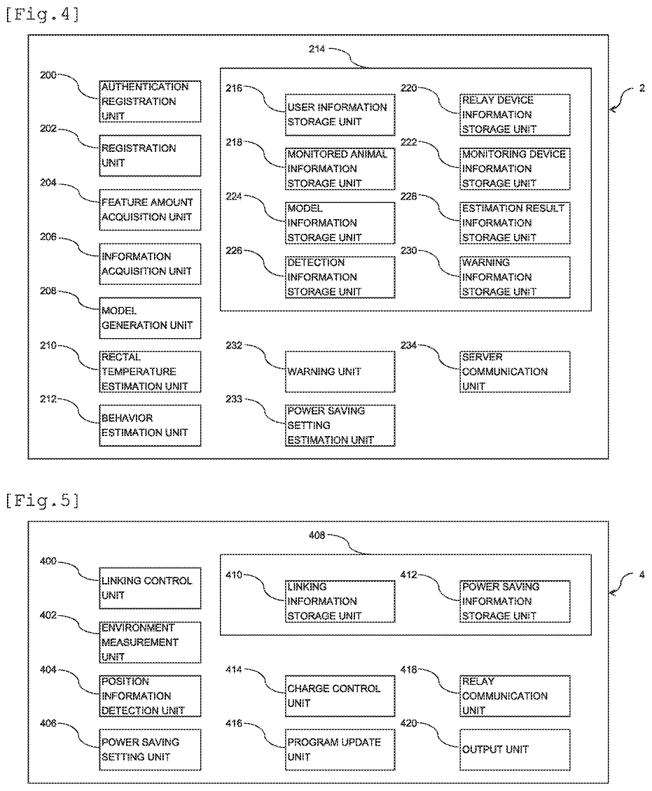

FIG. 4 illustrates an example of a functional configuration of the server according to the present embodiment.

The server 2 includes an authentication registration unit 200 that executes user authentication, registration of each device, and the like, a registration unit 202 that registers the monitored animal 6, a feature amount acquisition unit 204 that acquires the feature amount of the monitored animal 6, an information acquisition unit 206 that acquires predetermined information, a model generation unit 208 that generates a predetermined learning model, a rectal temperature estimation unit 210 that estimates the rectal temperature of the monitored animal 6, a behavior estimation unit 212 that estimates the behavior of the monitored animal 6, a server storage unit 214 that stores various types of information, a warning unit 232 that notifies the information terminal 7 and the like of a predetermined warning, a power saving setting estimation unit 233 that outputs a condition for executing power saving control of the monitoring device 5, and a server communication unit 234 that communicates with the relay device 4, the information terminal 7, and the like. In addition, the server storage unit 214 includes a user information storage unit 216 that stores information regarding the user, a monitored animal information storage unit 218 that stores information regarding the monitored animal 6, a relay device information storage unit 220 that stores information regarding the relay device 4, a monitoring device information storage unit 222 that stores information regarding the monitoring device 5, a model information storage unit 224 that stores the learning model, a detection information storage unit 226 that stores predetermined detection information by the monitoring device 5, an estimation result information storage unit 228 that stores information regarding various estimation results related to the monitored animal 6, and a warning information storage unit 230 that stores information regarding whether the warning unit 232 issues a warning or the like.

### (Authentication Registration Unit 200)

The authentication registration unit 200 identifies each of the relay device 4, the monitoring device 5, and the information terminal 7, and sets each device to a state capable of using the server 2. That is, the authentication registration unit 200 authenticates each device as a device capable of using the server 2, and registers each device by storing predetermined information in a predetermined storage unit of the server storage unit 214. Specifically, the authentication registration unit 200 receives relay device identification information (relay device ID) that identifies the relay device 4 from the relay device 4, stores the information in the relay device information storage unit 220, and registers the information as a relay device managed in the server 2. In addition, the authentication registration unit 200 receives monitoring device identification information (monitoring device ID) that identifies the monitoring device 5 from the monitoring device 5, stores the information in the monitoring device information storage unit 222, and registers the information as a monitoring device managed in the server 2.

Furthermore, the authentication registration unit 200 receives information terminal identification information (information terminal ID) that identifies the information terminal 7 from the information terminal 7, stores the information in the user information storage unit 216, and registers the information as an information terminal managed in the server 2. In this case, the authentication registration unit 200 creates account information of the user who uses the information terminal 7. The authentication registration unit 200 stores the account information (for example, a user ID, a password, and the like) of the user and the information regarding the user in the user information storage unit 216 in association with a user identifier that uniquely identifies the user. Through the above processing, the authentication registration unit 200 sets each device to the state capable of using the server 2.

### (Registration Unit 202)

The registration unit 202 acquires the information regarding the monitored animal 6 via the information terminal 7, and registers the information as the monitored animal as a monitoring target in the server 2. Specifically, the registration unit 202 acquires information such as a name identifying the monitored animal 6, as the information regarding the monitored animal 6, from the information terminal 7, and stores the acquired information in the monitored animal information storage unit 218 in association with a monitored animal identifier (monitored animal ID) that uniquely identifies the monitored animal 6. As a result, the registration unit 202 registers the monitored animal 6 in the server 2.

### (Feature Amount Acquisition Unit 204)

The feature amount acquisition unit 204 acquires the feature amount of the monitored animal 6. The feature amount acquisition unit 204 acquires the feature amount of the monitored animal 6 via the information terminal 7. For example, the feature amount acquisition unit 204 acquires, from the information terminal 7, the feature amount of the monitored animal 6 input to the information terminal 7 by the user. The feature amount acquisition unit 204 supplies the acquired feature amount to the model generation unit 208, the rectal temperature estimation unit 210, the behavior estimation unit 212, and/or the monitored animal information storage unit 218.

Examples of the feature amount include information regarding the kind of the monitored animal 6, information regarding the size of the monitored animal 6 (e.g., a chest circumference, a neck circumference, a height size, a weight, and the like), information regarding a hair length of the monitored animal 6, information regarding the hair amount of the monitored animal 6, information regarding a hair length at a predetermined position of the body of the monitored animal 6, information regarding a hair quality of the monitored animal 6 (e.g., a single coat, a double coat, and the like), and/or information regarding an age of the monitored animal 6. Note that the predetermined position in the information regarding the hair length at the predetermined position of the body of the monitored animal 6 is preferably a position where, for example, the body surface temperature detection unit 500 is attached.

Note that the feature amount acquisition unit 204 may correct the feature amount stored in the monitored animal information storage unit 218 according to an instruction or the like of the user received via the information terminal 7. In addition, the feature amount acquisition unit 204 may automatically update the feature amount that changes with time, such as the age of the monitored animal 6. As an example, a case where the monitored animal 6 is a pet of the user and is a dog (dog breed: miniature golden doodle) will be described. In this case, the feature amount acquisition unit 204 acquires, as the feature amount, information on the dog breed that is the kind of the pet, information on the weight that is the size of the pet, information on the hair length of the pet, and the like. For the hair length, the user measures the hair length of the pet, and the feature amount acquisition unit 204 acquires the measured length input via an input unit 700 of the information terminal 7. Note that the hair length of the pet can change depending on the season (for example, short in summer, and longer in winter than in summer). Therefore, the feature amount acquisition unit 204 may acquire the feature amount that changes with time such as the season at every predetermined timing (for example, a predetermined month) via the input unit 700 of the information terminal 7.

### (Information Acquisition Unit 206)

The information acquisition unit 206 acquires various types of information regarding the monitored animal 6 and/or information regarding an environment in which the monitored animal 6 is present, and the like. Specifically, the information acquisition unit 206 acquires, from the monitoring device 5, information regarding the body surface temperature of the monitored animal 6 detected by the monitoring device 5 and/or information regarding the motion of the monitored animal 6 detected by the monitoring device 5, and the like. In addition, the information acquisition unit 206 acquires, from the relay device 4, environmental information regarding an environment around the monitored animal 6 detected by the relay device 4 and/or information regarding a position of the monitoring device 5 with the relay device 4 as a base point. The information acquisition unit 206 stores each piece of acquired information in the detection information storage unit 226, and further supplies the information to the rectal temperature estimation unit 210, the behavior estimation unit 212, and the warning unit 232.

### (Model Generation Unit 208)

The model generation unit 208 generates a rectal temperature estimation model for estimating the rectal temperature of the monitored animal 6, a behavior estimation model for estimating the behavior of the monitored animal 6, and/or a power saving setting estimation model for determining the condition for causing the monitoring device 5 to execute efficient power saving control. The model generation unit 208 stores the generated model in the model information storage unit 224. Note that known machine learning such as deep learning can be used as machine learning in the model generation unit 208 described below.

### (Model Generation Unit 208: Rectal Temperature Estimation Model)

The animal monitoring system 1 uses the rectal temperature estimation model (learning model) prepared by learning in advance the feature amount (for example, the body surface temperature and the like) of a predetermined animal (monitored animal) and the rectal temperature of the animal to calculate the estimated rectal temperature of the animal. In the learning, a relationship between the body surface temperature and the rectal temperature is learned using predetermined training data.

Specifically, the model generation unit 208 uses, for each kind of predetermined animal, a combination of the body surface temperature of the animal or the body surface temperature around the neck of the animal, the feature amount of the animal, the rectal temperature of the animal detected simultaneously with the body surface temperature of the animal or the body surface temperature around the neck of the animal, and/or the environmental information around the animal, as rectal temperature training data, and performs learning by a machine learning algorithm using, for example, a gradient method. As a result, the model generation unit 208 generates the rectal temperature estimation model that causes a processor of the server 2 to function to output the rectal temperature of the animal when the body surface temperature of the animal, the feature amount of the animal, and/or the environmental information around the animal are/is input.

In addition, after generating the rectal temperature estimation model once, the model generation unit 208 may add the body surface temperature of a specific animal (for example, the pet of the user or the like) or the body surface temperature around the neck of the animal, the feature amount of the animal, the rectal temperature of the animal detected simultaneously with the body surface temperature of the animal or the body surface temperature around the neck of the animal, and/or the environmental information around the animal, to the rectal temperature training data, and generate the rectal temperature estimation model again. The model generation unit 208 may generate the rectal temperature estimation model again every time the feature amount of the specific animal changes (for example, every birthday, every season in which the hair amount changes, and the like).

Here, from the viewpoint of accurate estimation of the rectal temperature, the model generation unit 208 preferably uses the rectal temperature training data including at least a combination of the body surface temperature of the animal, particularly the body surface temperature around the neck (preferably the throat side) of the animal and the rectal temperature of the animal when the body surface temperature is detected.

Note that the environmental information is information regarding the environment of a predetermined area where the relay device 4 is installed and where the monitored animal 6 is present. For example, the environmental information includes information regarding temperature, humidity, illuminance, and/or sound of the area. In the present embodiment, the environmental information is preferably information regarding the temperature and the humidity from the viewpoint of easiness of acquisition and/or simplification of the configuration of the relay device 4.

### (Model Generation Unit 208: Behavior Estimation Model)

The animal monitoring system 1 uses the behavior estimation model (learning model) prepared by learning in advance the motion of a predetermined animal (monitored animal) and the behavior corresponding to this motion to calculate information regarding the estimated behavior of the animal. In the learning, a relationship between the motion and the behavior is learned using predetermined training data.

Specifically, the model generation unit 208 uses, for each kind of predetermined animal (monitored animal), a combination of motion information indicating the motion of the animal (for example, a detection value by a motion detection unit 502 to be described later) and behavior information indicating the behavior of the animal corresponding to this motion, as behavior training data, and performs learning by a machine learning algorithm using, for example, a gradient method. As a result, the model generation unit 208 generates the behavior estimation model that causes the processor of the server 2 to function to output the behavior information of the animal when the motion information of the animal is input. The behavior information is information accumulated by observing the behaviors of a large number of animals (Not only the animal owned by the user but also animals owned by other users are included), and may be information collected together with the feature amount such as the kind and size of each animal. In addition, after generating the behavior estimation model once, the model generation unit 208 may add the motion information of the motion of a specific animal (for example, the pet of the user or the like) and the behavior information (This behavior information is, for example, information obtained by the user observing the user's animal) of the behavior corresponding to this motion, to the behavior training data, and generate the behavior estimation model again. Note that the behavior training data may include information on the rectal temperature estimated by the rectal temperature estimation unit 210, the environmental information such as the temperature and humidity measured by the relay device 4, and/or information on a signal intensity transmitted by the monitoring device 5 and received by the relay device 4. In this case, when the motion information of the animal, the rectal temperature, the environmental information, and/or the signal intensity are/is input, the behavior estimation model outputs the behavior information of the animal.

In addition, the model generation unit 208 may acquire the behavior information obtained by the user himself/herself observing his/her own animal (pet) as one piece of behavior training data as described below. That is, the model generation unit 208 acquires information recorded by the user together with a detection result by the motion detection unit 502 to be described later regarding the behavior of the pet via the information terminal 7 and incorporates the acquired information into the behavior training data. As a result, the behavior training data can include the behavior information specific to the pet of the specific user.

That is, when the user finds a predetermined behavior of the pet, the model generation unit 208 acquires behavior information corresponding to this behavior via the information terminal 7. For example, the model generation unit 208 causes an output unit 702 of the information terminal 7 to output a plurality of options that allow for selection of one behavior from a plurality of predetermined behaviors, and determines that the behavior corresponding to the option selected by the user, received via the input unit 700, is a behavior actually performed by the pet. The model generation unit 208 incorporates the detection result by the motion detection unit 502 when the option is selected, into the behavior training data as a detection result at the time of the behavior.

As a result, information in which a specific behavior in the area where the pet is present (for example, a user's room) is associated with motion information regarding a motion of the pet at the time of the specific behavior is incorporated into the behavior training data. Therefore, for example, with regard to an excretion (toilet) behavior of the pet, the behavior training data includes the detection result by the motion detection unit 502 (for example, a detection result indicating a posture of the pet detected by a 3-axis acceleration sensor or a 3-axis gyro sensor) at the time of the behavior detected according to a toilet position specific to the area where the pet is present and a direction in which the pet is directed during excretion. Thus, the model generation unit 208 can generate the behavior estimation model adapted to the pet with high accuracy. As an example, it is possible to determine whether the pet is excreting or eating according to a direction specified by a direction in which a cage is placed in the area where the pet is present.

### (Model Generation Unit 208: Power Saving Setting Estimation Model)

The animal monitoring system 1 uses the power saving setting estimation model (learning model) prepared by learning in advance the motion of the monitored animal, information regarding charging of the monitoring device 5, and/or information regarding power consumption thereof to calculate the condition for executing the power saving control of the monitoring device 5. In the learning, a relationship between the motion and the like and the condition is learned using predetermined training data. Note that the power saving setting estimation model is a model that improves the efficiency of power consumption within a range not impairing the detection accuracy of the body surface temperature and the motion of the monitored animal 6 by the monitoring device 5, and the power saving setting estimation model can also output a threshold of optimal acceleration (acceleration detected by the motion detection unit 502) for stopping the body surface temperature detection unit 500 or the motion detection unit 502 of the monitoring device 5 to be described later by inputting information such as the motion of the monitored animal 6.

Specifically, the model generation unit 208 uses a combination of the motion information of the monitored animal 6 wearing the monitoring device 5, the body surface temperature of the monitored animal 6, the information regarding the behavior obtained by the user observing the monitored animal 6, the information regarding the charging of the monitoring device 5, and/or the information regarding the power consumption in the monitoring device 5, as power saving setting training data, and performs learning by a machine learning algorithm using, for example, a gradient method. As a result, the model generation unit 208 generates the power saving setting estimation model that causes the processor of the server 2 to function to output the condition for executing the power saving control of the monitoring device 5 when the motion information of the monitored animal 6, the information regarding the charging, and/or the information regarding the power consumption are/is input. Note that this condition is, for example, a condition for activating or stopping each constituent member included in the monitoring device 5, and a condition for determining an optimal sampling frequency or the like in a constituent member for detecting the body surface temperature or the like of the monitored animal 6.

Note that the model generation unit 208 may generate the rectal temperature estimation model using, as the rectal temperature training data, the information of the monitored animal 6 acquired by the monitoring device 5 (the information regarding the body surface temperature and the motion), information of a monitored animal different from the monitored animal 6 (for example, a monitored animal owned by a user different from the user having the monitored animal 6), and/or the information and/or the feature amount of the monitored animal acquired outside the animal monitoring system 1 or in the animal monitoring system 1 different from this animal monitoring system 1. The same applies to the behavior estimation model and the power saving setting estimation model.

### (Rectal Temperature Estimation Unit 210)

The rectal temperature estimation unit 210 applies the body surface temperature detected by the body surface temperature detection unit 500 to the rectal temperature estimation model to estimate the rectal temperature of the monitored animal 6. That is, the rectal temperature estimation unit 210 calculates the rectal temperature of the animal by inference processing using the rectal temperature estimation model constructed by learning the body surface temperature data of a large number of animals and the rectal temperatures of the animals by using the machine learning algorithm using the gradient method or the like. For example, the rectal temperature estimation unit 210 calculates the rectal temperature of the animal using the body surface temperature of the animal acquired by the monitoring device 5, the feature amount of the animal, and/or the environmental information around the animal, and the rectal temperature estimation model. That is, the rectal temperature estimation unit 210 outputs an estimated value of the rectal temperature of the monitored animal 6 by applying the body surface temperature detected by the body surface temperature detection unit 500 to the rectal temperature estimation model that receives the combination of at least the body surface temperature of the monitored animal 6 and the rectal temperature of the monitored animal, as the rectal temperature training data, and outputs the rectal temperature of the monitored animal from at least the body surface temperature of the monitored animal by machine learning using the rectal temperature training data. The rectal temperature estimation unit 210 stores the output rectal temperature in the estimation result information storage unit 228. In addition, the rectal temperature estimation unit 210 supplies the output rectal temperature to the warning unit 232. Note that the rectal temperature estimation unit 210 can output the rectal temperature as the estimated value at predetermined time intervals (for example, at intervals of one second or the like).

### (Behavior Estimation Unit 212)

The behavior estimation unit 212 estimates the behavior of the monitored animal 6 on the basis of the motion information detected by the motion detection unit 502 to be described later. Specifically, the behavior estimation unit 212 applies the motion information indicating the motion of the monitored animal 6 detected by the monitoring device 5 to the behavior estimation model to estimate the behavior of the monitored animal 6. That is, the behavior estimation unit 212 calculates the behavior information of the estimated behavior of the animal by inference processing using the behavior estimation model constructed by learning the motion information of a large number of animals and the behavior information indicating the behaviors corresponding to the motion information of the animals by using the machine learning algorithm using the gradient method or the like. For example, the behavior estimation unit 212 calculates the behavior information of the animal using the body surface temperature and/or the motion of the animal acquired by the monitoring device 5, the feature amount of the animal, and/or the environmental information around the animal, and the behavior estimation model. That is, the behavior estimation unit 212 outputs the behavior information of the estimated behavior of the monitored animal 6 by applying the motion information of the motion detected by the monitoring device 5 to the behavior estimation model that receives the combination of at least the motion information of the motion of the monitored animal 6 and the behavior information of the behavior corresponding to this motion, as the behavior training data, and outputs the behavior information of the monitored animal from at least the motion information of the monitored animal by machine learning using the behavior training data. The behavior estimation unit 212 stores the output behavior information in the estimation result information storage unit 228. In addition, the behavior estimation unit 212 supplies the behavior information to the warning unit 232 and a power saving control unit 508 of the monitoring device 5. Note that the behavior estimation unit 212 can output the behavior information at predetermined time intervals (for example, at intervals of one second or the like). In addition, the behavior estimation unit 212 may estimate that the monitored animal 6 has a behavior when a detection value of a predetermined sensor in the motion detection unit 502 to be described later exceeds a predetermined value, and that the monitored animal 6 has no behavior when the detection value is equal to or lower than the predetermined value.

Note that the behavior information is information indicating a behavior performed by the animal in life, and is information indicating, for example, eating, excretion, sleeping (including falling asleep and waking up behaviors), taking a break or resting (stationary state), grooming, drinking water, and/or exercise (walking movement, running movement, jumping movement, and the like) of the monitored animal 6.

### (Warning Unit 232)

The warning unit 232 outputs warning information on the basis of condition information stored in the warning information storage unit 230 to be described later. The warning unit 232 refers to the condition information stored in the warning information storage unit 230 and determines whether to output the warning information. Specifically, the warning unit 232 outputs the warning information in a case where the rectal temperature estimated by the rectal temperature estimation unit 210 is a temperature equal to or higher than a predetermined threshold. In addition, the warning unit 232 may output the warning information on the basis of the behavior information received from the behavior estimation unit 212. The warning unit 232 outputs the warning information in a case where the behavior information received from the behavior estimation unit 212 matches behavior information as the condition information. Here, the behavior information as the condition information is an index requiring attention of the user as an index regarding a health condition of the monitored animal 6. As an example, the behavior information as the condition information is information indicating lengths of various behaviors (behaviors such as eating, excretion, sleeping, and standing still), a time between one behavior (for example, eating or the like) and another behavior (for example, excretion or the like), and/or other behaviors that cannot be normally taken. Furthermore, the warning unit 232 can output the warning information on the basis of the environmental information acquired by the information acquisition unit 206. The warning unit 232 outputs the warning information in a case where the environmental information acquired by the information acquisition unit 206 matches environmental information as the condition information. Here, the environmental information as the condition information is, for example, a threshold determined in advance for the temperature, humidity, and the like around the monitored animal 6. The warning unit 232 outputs the warning information in a case where the environmental information is in a predetermined state (for example, a state in which the temperature and/or the humidity are/is equal to or higher than the predetermined threshold).

The warning unit 232 supplies the warning information to the relay device 4 and/or the information terminal 7. The warning unit 232 supplies the warning information to the information terminal 7 by e-mail, short message service (SMS), or the like. In addition, the warning unit 232 may supply the warning information to a predetermined application installed in advance in the information terminal 7, and the application may output warning contents corresponding to the warning information by push notification in a user-perceptible manner. The relay device 4 and/or the information terminal 7 receiving the warning information output (s) the warning information in a user-perceptible manner. The relay device 4 and/or the information terminal 7 output (s) the warning information in a format such as text, image, and/or audio, for example.

### (Server Storage Unit 214)

The server storage unit 214 stores an operating system (OS) and various types of information regarding the animal monitoring system 1. The server storage unit 214 supplies predetermined information to a predetermined constituent member in response to a request from another constituent member. In addition, the server storage unit 214 stores information supplied from another constituent member in a predetermined storage unit.

### (Server Storage Unit 214: User Information Storage Unit 216)

The user information storage unit 216 stores the information regarding the user in association with the user identifier. The information regarding the user includes a username, a user ID for an account of the user, a password for the account, information regarding the information terminal 7 used by the user (for example, the information terminal identification information that identifies the information terminal 7, a telephone number, and the like), and/or other personal information of the user such as a mail address.

### (Server Storage Unit 214: Monitored Animal Information Storage Unit 218)

The monitored animal information storage unit 218 stores the information regarding the monitored animal 6 in association with the monitored animal identifier of the monitored animal 6 and the user identifier of the user who owns the monitored animal 6. The information regarding the monitored animal 6 is information such as a name of the monitored animal 6, the feature amount of the monitored animal 6, and/or an image showing the monitored animal 6.

### (Server Storage Unit 214: Relay Device Information Storage Unit 220)

The relay device information storage unit 220 stores the information regarding the relay device 4 in association with the relay device identification information of the relay device 4 that is linked with the monitoring device 5 attached to the monitored animal 6. The information regarding the relay device 4 includes a manufacturing number of the relay device 4, information regarding the information terminal 7 with which the relay device 4 is associated (for example, the information terminal identification information of the information terminal 7 and the information regarding the user of the information terminal 7 (e.g., the user identifier)), and/or a MAC address.

### (Server Storage Unit 214: Monitoring Device Information Storage Unit 222)

The monitoring device information storage unit 222 stores the information regarding the monitoring device 5 registered in the animal monitoring system 1 in association with the monitoring device identification information of the monitoring device 5. The information regarding the monitoring device 5 is information such as the user identifier of the user of the information terminal 7 linked with the monitoring device 5, a type of the monitoring device (for example, a type indicating at which position of the body of the monitored animal 6 the device is to be attached), contents of the power saving control in a case where the monitoring device is subjected to the power saving control, the condition for executing the power saving control (for example, a value of acceleration applied to the monitoring device 5, or the like), a charging and discharging history, and/or a power consumption history.

### (Server Storage Unit 214: Model Information Storage Unit 224)

The model information storage unit 224 stores the learning model used in the animal monitoring system 1. The learning model is, for example, the rectal temperature estimation model, the behavior estimation model, and/or the power saving setting estimation model.

### (Server Storage Unit 214: Detection Information Storage Unit 226)

The detection information storage unit 226 stores various types of information detected by the monitoring device 5 and acquired by the information acquisition unit 206. The detection information storage unit 226 stores the various types of information in association with the monitoring device identification information of the monitoring device 5 and detection dates and times of the various types of information. The various types of information include the information regarding the body surface temperature of the monitored animal 6 and/or the motion of the monitored animal 6 detected by the monitoring device 5, and the like. In addition, the detection information storage unit 226 stores the environmental information detected by the relay device 4. The detection information storage unit 226 stores the environmental information in association with the relay device identification information of the relay device 4 and a detection date and time of the environmental information.

### (Server Storage Unit 214: Estimation Result Information Storage Unit 228)

The estimation result information storage unit 228 stores the rectal temperature of the monitored animal 6 output by the rectal temperature estimation unit 210 and/or the behavior information of the monitored animal 6 estimated by the behavior estimation unit 212. The estimation result information storage unit 228 stores the rectal temperature and/or the behavior information of the monitored animal 6 identified by the monitored animal identifier, in association with the monitored animal identifier. In addition, the estimation result information storage unit 228 can store the rectal temperature calculated on the basis of the body surface temperature of the monitored animal 6 in association with a detection date and time of the body surface temperature by the monitoring device 5. Similarly, the estimation result information storage unit 228 can store the behavior information estimated on the basis of the motion of the monitored animal 6 in association with a detection date and time of the motion by the monitoring device 5. Note that the detection date and time may also include year, month, day, hour, minute, and second of detection.

### (Server Storage Unit 214: Warning Information Storage Unit 230)

The warning information storage unit 230 stores the condition information indicating a condition under which the warning unit 232 outputs the warning information. Specifically, the warning information storage unit 230 stores, as the condition information, a rectal temperature threshold serving as a reference for the warning unit 232 to output the warning information. The warning information storage unit 230 also stores, as the condition information, the behavior information and/or the environmental information serving as the condition under which the warning unit 232 outputs the warning information.

### (Power Saving Setting Estimation Unit 233)

The power saving setting estimation unit 233 applies the motion information indicating the motion of the monitored animal 6 detected by the monitoring device 5, the information regarding the charging of the monitoring device 5, and/or the information regarding the power consumption in the monitoring device 5, to the power saving setting estimation model, to output the condition for causing the monitoring device 5 to execute efficient power saving control. That is, the power saving setting estimation unit 233 outputs the condition for causing the monitoring device 5 to execute the power saving control by inference processing using the power saving setting estimation model constructed by learning the motion information of a large number of animals, the information regarding the charging, and/or the information regarding the power consumption by using the machine learning algorithm using the gradient method or the like.

For example, the power saving setting estimation unit 233 outputs the condition for causing the monitoring device 5 to execute the power saving control by using the body surface temperature and/or the motion of the animal acquired by the monitoring device 5, the information regarding the charging of the monitoring device 5, and/or the information regarding the power consumption of the monitoring device 5, and the power saving setting estimation model. That is, the power saving setting estimation unit 233 outputs the condition for executing the power saving control by applying the motion information of the motion detected by the monitoring device 5 to the power saving setting estimation model that receives at least the motion information of the motion of the monitored animal 6 and the information regarding the power consumption, as the power saving setting training data, and outputs the condition for causing the monitoring device 5 to execute the efficient power saving control from the motion information of the monitored animal by machine learning using the power saving setting training data.

For example, the power saving setting estimation model may be a model that compares the power consumption of the monitoring device 5 during motion indicated by one piece of motion information with the power consumption of the monitoring device 5 during motion indicated by another piece of motion information when the one piece of motion information is input, and outputs a command to, for example, stop operation of a predetermined constituent member of the monitoring device 5, as the condition for executing the power saving control of the monitoring device 5, during the motion indicated by the other piece of motion information when the power consumption during the motion indicated by the one piece of motion information is larger than the power consumption during the motion indicated by the other piece of motion information. That is, the power saving control of the monitoring device 5 is started when the behavior of the monitored animal 6 is low (for example, during sleeping, resting, and the like), and the power saving control of the monitoring device 5 is stopped when the behavior is active (for example, during exercise or eating).

The power saving setting estimation unit 233 stores the output condition in the monitoring device information storage unit 222. In addition, the power saving setting estimation unit 233 supplies the output condition to the relay device 4. When receiving the condition, a power saving setting unit 406 of the relay device 4 starts the power saving control of the monitoring device 5 according to the condition. The power saving control that the relay device 4 causes the power saving control unit 508 of the monitoring device 5 to execute is, for example, control of activating or stopping each constituent member included in the monitoring device 5, and reducing a sampling frequency for information detection in a member that detects predetermined information. Note that the power saving setting estimation unit 233 can execute the control according to the condition of the power saving control at predetermined time intervals (for example, at intervals of one second or the like).

### (Server Communication Unit 234)

The server communication unit 234 communicates with the relay device 4 and the information terminal 7 via the communication network 3 so as to enable bidirectional communication. Note that each constituent member included in the server 2 acquires predetermined information from the relay device 4 and/or the information terminal 7 via the server communication unit 234, and supplies predetermined information to a predetermined constituent member of the relay device 4 and/or the information terminal 7 via the server communication unit 234.

### [Details of Relay Device 4]

FIG. 5 illustrates an example of a functional configuration of the relay device according to the present embodiment.

The relay device 4 includes a linking control unit 400 that controls linking processing with the monitoring device 5 and the information terminal 7, an environment measurement unit 402 that measures a situation of a surrounding environment, a position information detection unit 404 that detects a position of the monitoring device 5, the power saving setting unit 406 that sets the monitoring device 5 to a power saving mode, a relay device storage unit 408 that stores various types of information, a charge control unit 414 that controls the charging of the monitoring device 5, a program update unit 416 that updates a program installed in the relay device 4 and/or the monitoring device 5, a relay communication unit 418 that communicates with the server 2 and the like, and an output unit 420 that outputs predetermined information. The relay device storage unit 408 includes a linking information storage unit 410 that stores information regarding the linking processing, and a power saving information storage unit 412 that stores information regarding the power saving control of the monitoring device 5. Note that the relay device 4 can be configured as a personal computer or an information terminal in which a general-purpose operating system is installed and operated, or can be configured using a predetermined microcomputer. In addition, each constituent member of the relay device 4 operates by receiving power supply from an external power source.

### (Linking Control Unit 400)

The linking control unit 400 sets the relay device 4 and the server 2, the relay device 4 and the monitoring device 5, and the relay device 4 and the information terminal 7 to a bidirectionally communicating state (hereinafter, such setting may be referred to as “linking”). That is, the linking control unit 400 establishes a communication connection among the server 2, the relay device 4, the monitoring device 5, and/or the information terminal 7.

### (Environment Measurement Unit 402)

The environment measurement unit 402 measures the situation regarding the environment around the relay device 4. Specifically, the environment measurement unit 402 measures the temperature, humidity, illuminance, oxygen concentration, carbon dioxide concentration, and/or sound, and the like of the surrounding environment. Therefore, the environment measurement unit 402 includes a temperature sensor, a humidity sensor, an illuminance sensor, a gas sensor, and/or an audio sensor, and the like. The environment measurement unit 402 is preferably configured to measure at least the temperature and the humidity from the viewpoint of easiness of configuration. The environment measurement unit 402 supplies the environmental information indicating the measured situation regarding the environment to the information acquisition unit 206 of the server 2.

Here, the environment measurement unit 402 is installed in a space where the monitored animal 6 is located and at a position separated from the body surface temperature detection unit 500 of the monitoring device 5 attached to the monitored animal 6. That is, since the environment measurement unit 402 is included in the relay device 4, the environment measurement unit 402 is at a position separated from the monitored animal 6.

Note that the environment measurement unit 402 can add a predetermined flag to the environmental information measured in a case where the relay device 4 and the monitoring device 5 are in the bidirectionally communicating state, and supply the environmental information to the information acquisition unit 206. This flag may be a flag indicating that the environmental information can be used in the model generation unit 208, the rectal temperature estimation unit 210, and the behavior estimation unit 212. The model generation unit 208, the rectal temperature estimation unit 210, and the behavior estimation unit 212 each execute predetermined processing using the environmental information to which the flag is added. As a result, it is possible to exclude the environmental information obtained when the monitoring device 5 is at a position not capable of communicating with the relay device 4 (when the monitored animal 6 moves away from the relay device 4 and the communication between the relay device 4 and the monitoring device 5 cannot be performed). While the information acquisition unit 206 receives the environmental information to which the flag is not added, the rectal temperature estimation unit 210 and the behavior estimation unit 212 may stop outputting the rectal temperature and the behavior information of the monitored animal 6, respectively. In addition, while the bidirectional communication cannot be performed between the relay device 4 and the monitoring device 5 (uncommunicable period), the relay communication unit 418 may notify the server 2 of the uncommunicable period, and the rectal temperature estimation unit 210 and the behavior estimation unit 212 may stop the respective processing during the uncommunicable period.

### (Position Information Detection Unit 404)

The position information detection unit 404 detects position information regarding the position of the monitoring device 5 relative to the relay device 4. The information regarding the position is information regarding a distance from the relay device 4 to the monitoring device 5 and/or a direction in which the monitoring device 5 exists with the relay device 4 as a base point. For example, the position information detection unit 404 estimates the distance from the relay device 4 to the monitoring device 5 and the direction of the monitoring device 5 on the basis of a signal intensity from a monitoring device communication unit 520 to be described later. That is, since the signal intensity transmitted by the monitoring device communication unit 520 is damped according to the distance, the position information detection unit 404 estimates the distance to the monitoring device 5 on the basis of the received signal intensity. In addition, the position information detection unit 404 may include a plurality of signal reception units, thereby estimating the direction of the monitoring device 5 relative to the relay device 4 on the basis of the signal intensities received by the respective signal reception units. The position information detection unit 404 supplies the detected position information of the monitoring device 5 to the information acquisition unit 206.

### (Power Saving Setting Unit 406)

The power saving setting unit 406 executes the power saving control of the monitoring device 5 on the basis of the condition (power saving execution condition) for executing the power saving control received from the power saving setting estimation unit 233 of the server 2. The power saving setting unit 406 may also directly receive the estimation result of the behavior estimation unit 212 and execute the power saving control of the monitoring device 5 on the basis of the received estimation result.

### (Relay Device Storage Unit 408)

The relay device storage unit 408 stores an OS and various types of information regarding the animal monitoring system 1. The relay device storage unit 408 supplies predetermined information to a predetermined constituent member in response to a request from another constituent member. In addition, the relay device storage unit 408 stores information supplied from another constituent member in a predetermined storage unit. Note that the relay device storage unit 408 can also store information regarding the relay device 4 itself (for example, the relay device identification information of the relay device 4, various setting information regarding operation setting of the relay device 4, and the like).

### (Relay Device Storage Unit 408: Linking Information Storage Unit 410)

The linking information storage unit 410 stores information regarding the monitoring device 5 and/or the information terminal 7 linked with the relay device 4. The linking information storage unit 410 stores the information regarding the monitoring device 5 in association with a monitoring device identifier of the monitoring device 5. The linking information storage unit 410 also stores the information regarding the information terminal 7 in association with an information terminal identifier of the information terminal 7. The information regarding the monitoring device 5 stored in the linking information storage unit 410 may be the same as the information stored in the monitoring device information storage unit 222.

### (Relay Device Storage Unit 408: Power Saving Information Storage Unit 412)

The power saving information storage unit 412 stores the information regarding the power saving control of the monitoring device 5 linked with the relay device 4. The information regarding the power saving control is the condition for causing the monitoring device 5 to execute the power saving control on the basis of a predetermined power saving mode and/or information regarding the predetermined power saving mode. Note that the power saving mode is a mode in a case where the power saving control is performed on the monitoring device 5, and a plurality of patterns of power saving modes can be set by setting a combination of the activation and stop or the like of each constituent member included in the monitoring device 5. Therefore, the information regarding the power saving mode may be information indicating the patterns (power saving setting data).

### (Charge Control Unit 414, Program Update Unit 416)

The charge control unit 414 controls charging of a battery included in the monitoring device 5 mounted on the mounting portion 42 of the relay device 4. In addition, the program update unit 416 updates the program installed in the relay device 4 and updates the program installed in the monitoring device 5 mounted on the mounting portion 42.

### (Relay Communication Unit 418)

The relay communication unit 418 communicates with the server 2 and the information terminal 7 via the communication network 3 so as to enable bidirectional communication. In addition, the relay communication unit 418 can establish a communication connection between the monitoring device 5 and the relay device 4 and/or between the information terminal 7 and the relay device 4 so as to enable bidirectional communication by short-range wireless communication. Note that each constituent member included in the relay device 4 acquires predetermined information from the server 2, the monitoring device 5, and/or the information terminal 7 via the relay communication unit 418, and supplies predetermined information to a predetermined constituent member of the server 2, the monitoring device 5, and/or the information terminal 7 via the relay communication unit 418. For example, the relay communication unit 418 supplies information received from the monitoring device 5 (the information indicating the body surface temperature of the monitored animal 6, the information indicating the motion thereof, and the like) to the server 2.

### (Output Unit 420)

The output unit 420 outputs a situation regarding the relay device 4 and/or the monitoring device 5 in a user-perceptible manner. For example, the output unit 420 outputs the warning information received from the warning unit 232. As an example, the output unit 420 is a display unit (a liquid crystal display unit, an organic EL display, or the like) that outputs text information or image information, and/or an audio output unit.

### [Details of Monitoring Device 5 and Information Terminal 7]

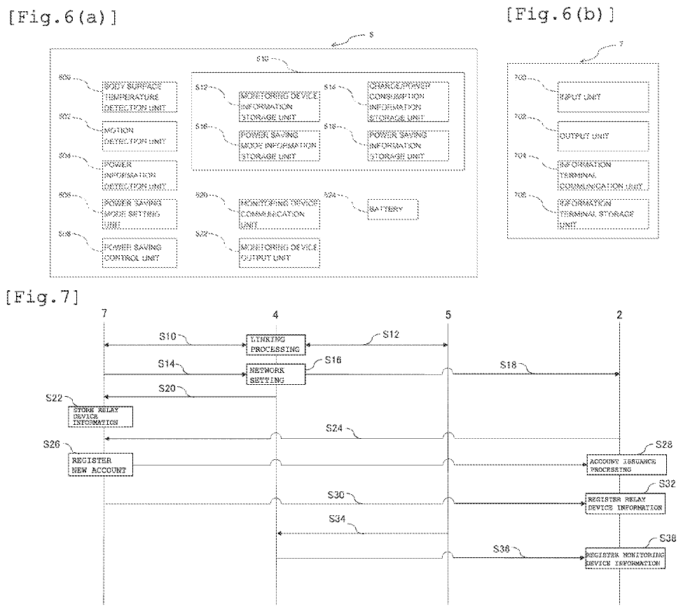

FIG. 6 illustrates an example of a functional configuration of the monitoring device and the information terminal according to the present embodiment. Specifically, FIG. 6(a) illustrates an outline of an example of the functional configuration of the monitoring device 5, and FIG. 6(b) illustrates an outline of an example of the functional configuration of the information terminal 7.

The monitoring device 5 detects the body surface temperature of the monitored animal 6 and detects the motion (movement) thereof. That is, as illustrated in FIG. 6(a), the monitoring device 5 includes the body surface temperature detection unit 500 that detects the body surface temperature of the monitored animal 6, the motion detection unit 502 that detects the motion of the monitored animal 6, a power information detection unit 504 that detects information regarding a battery 524 of the monitoring device 5, a power saving mode setting unit 506 that sets contents of the power saving mode of the monitoring device 5, the power saving control unit 508 that executes the power saving control, a monitoring device storage unit 510 that stores various types of information, the monitoring device communication unit 520 that communicates with the outside, the monitoring device output unit 522 that outputs predetermined information, and the battery 524 that supplies power to each constituent member of the monitoring device 5. In addition, the monitoring device storage unit 510 includes a monitoring device information storage unit 512 that stores information regarding the monitoring device 5, a charge/power consumption information storage unit 514 that stores information regarding charging and power consumption of the battery 524, a power saving mode information storage unit 516 that stores information regarding the power saving mode, and a power saving information storage unit 518 that stores information regarding the condition for executing the power saving control.

Note that each constituent member of the monitoring device 5 can be configured by a microcomputer except for the body surface temperature detection unit 500 and the motion detection unit 502, and can operate by power supply from the battery 524. In addition, as an example, the monitoring device 5 can be configured as a collar-type device attached around the neck of the monitored animal 6. The monitoring device 5 may be a device attached to another portion of the body of the monitored animal 6 as long as the monitoring device 5 can detect the body surface temperature and the motion of the monitored animal 6 and the monitored animal 6 hardly feels annoying.

### (Body Surface Temperature Detection Unit 500)

The body surface temperature detection unit 500 is attached to the monitored animal 6 using an auxiliary tool such as a belt and detects the body surface temperature of the monitored animal 6. The body surface temperature detection unit 500 is attached to the body surface of the monitored animal 6. The body surface temperature detection unit 500 is preferably attached at a position excluding the anal portion of the monitored animal 6, more preferably around the neck, and still more preferably around the throat. The body surface temperature detection unit 500 is not particularly limited as long as it is a sensor or the like capable of measuring the body surface temperature, and is a small sensor. For example, the body surface temperature detection unit 500 can be configured using a thermistor. The body surface temperature detection unit 500 according to the present embodiment can be configured using a thermistor instead of an infrared ray sensor, and thus can be miniaturized, and can reduce annoying feeling of the monitored animal 6. The body surface temperature detection unit 500 supplies the information on the detected body surface temperature (body surface temperature information) to the information acquisition unit 206 and the detection information storage unit 226 via the relay communication unit 418 of the relay device 4.

### (Motion Detection Unit 502)

The motion detection unit 502 is attached to the monitored animal 6 and detects the motion and/or a direction of the monitored animal 6. For example, the motion detection unit 502 can be configured using a 9-axis motion sensor that measures each of acceleration, angular velocity, and geomagnetism in three axes. The 9-axis motion sensor includes a 3-axis acceleration sensor, a 3-axis gyro sensor (3-axis angular velocity sensor), and a 3-axis geomagnetic sensor (3-axis magnetic sensor). The 3-axis gyro sensor is, for example, a MEMS gyro sensor. In the motion detection unit 502, a threshold for activating each of the 3-axis acceleration sensor, the 3-axis gyro sensor, and the 3-axis geomagnetic sensor can be set for each sensor. In addition, the motion detection unit 502 is not limited to the 9-axis motion sensor, and may be other motion sensor.

The 3-axis acceleration sensor and the 3-axis gyro sensor detect various motions (movements) of the monitored animal 6. That is, the 3-axis acceleration sensor and the 3-axis gyro sensor detect acceleration and angular velocity of a vibration (shaking) state applied to the sensors, respectively, and output voltage values of a vibration waveform corresponding to the magnitude of the vibration (that is, the acceleration and the angular velocity). For example, the 3-axis acceleration sensor outputs a voltage value proportional to the acceleration corresponding to the magnitude of the vibration applied in three-axis directions orthogonal to each other (an X-axis direction, a Y-axis direction, and a Z-axis direction), that is, the magnitude of the acceleration applied to three axes in front-rear, left-right, and vertical directions. In addition, the 3-axis gyro sensor outputs a voltage value proportional to the magnitude of the angular velocity around the three axes. Furthermore, the 3-axis geomagnetic sensor detects which direction the monitored animal 6 is facing and what posture the monitored animal 6 is taking as the posture (that is, the direction and inclination) of the monitored animal 6, and outputs a voltage value corresponding to the direction or the posture.

The motion detection unit 502 supplies the detection values individually detected by the 3-axis acceleration sensor, the 3-axis gyro sensor, and the 3-axis geomagnetic sensor (9-axis sensor), as the motion information, to the information acquisition unit 206 of the server 2 via the relay device 4. The motion detection unit 502 supplies the motion information to the information acquisition unit 206 at predetermined time intervals (for example, at intervals of one second, at intervals of five seconds, or the like) regardless of the presence or absence of a predetermined instruction from the information terminal 7. The information acquisition unit 206 stores the detection values detected by the motion detection unit 502 in the detection information storage unit 226 in association with the monitoring device identification information and the detection date and time. The motion detection unit 502 also supplies the detected detection values as the motion information to the power saving setting estimation unit 233 via the relay communication unit 418 of the relay device 4.

Note that the motion information included in the behavior training data used by the model generation unit 208 of the server 2 includes at least the detection value detected by the 3-axis acceleration sensor. The behavior training data may also include the detection value (s) detected by the 3-axis gyro sensor and/or the 3-axis geomagnetic sensor.

### (Power Information Detection Unit 504)

The power information detection unit 504 detects the information regarding the charging and discharging of the battery 524 and the information regarding the power consumption. The power information detection unit 504 detects these pieces of information at predetermined time intervals (for example, one second, five seconds, or the like). The power information detection unit 504 supplies the detected information to the power saving setting estimation unit 233 and the charge/power consumption information storage unit 514.

### (Power Saving Mode Setting Unit 506)

The power saving mode setting unit 506 sets the contents of the power saving mode of the monitoring device 5 defining activation, stop, and/or a sampling frequency of each sensor constituting the motion detection unit 502 on the basis of the detection result of any one of the 3-axis acceleration sensor, the 3-axis gyro sensor, and the 3-axis geomagnetic sensor. For example, in a case where a predetermined acceleration value is set as the threshold for activating each sensor of the 9-axis motion sensor, the power saving mode setting unit 506 sets the power saving mode to contents in which each sensor (however, excluding the 3-axis acceleration sensor) is controlled such that each sensor excluding the 3-axis acceleration sensor is stopped when the detection result of the 3-axis acceleration sensor falls below the predetermined acceleration value, and such that each sensor excluding the 3-axis acceleration sensor is activated when the detection result is equal to or higher than the predetermined acceleration. The power saving mode setting unit 506 supplies the power saving mode indicating the set contents to the power saving control unit 508 and the power saving mode information storage unit 516.

### (Power Saving Control Unit 508)

The power saving control unit 508 executes the power saving control of the body surface temperature detection unit 500 and/or the motion detection unit 502 of the monitoring device 5 by the following three methods, for example.

### (Method 1)

The power saving control unit 508 executes the power saving control on the basis of the estimation result of the behavior estimation unit 212 of the server 2. That is, the power saving control unit 508 executes the power saving control in a case where the estimation result of the behavior estimation unit 212 indicates a state in which the behavior of the monitored animal 6 is not active (For example, the state is a substantially stationary state such as a sleeping state or a resting state, and is referred to as an “inactive state”). That is, when the estimation result indicates that the monitored animal 6 is in a sleeping state or a resting state, the power saving setting estimation unit 233 of the server 2 outputs the condition (power saving execution condition) for causing the monitoring device 5 to execute the power saving control. The power saving setting estimation unit 233 supplies the power saving execution condition to the power saving setting unit 406 of the relay device 4. The power saving setting unit 406 controls the power saving control unit 508 on the basis of the power saving execution condition, and the power saving control unit 508 starts the power saving control of the monitoring device 5.

Note that the “substantially stationary state” includes not only a state in which the monitored animal 6 is literally stationary, but also a state in which the behavior estimated by the behavior estimation unit 212 on the basis of the motion detection by the motion detection unit 502 is a behavior with almost no movement or motion of the monitored animal 6 (a sleeping state, a resting state, a lying state, a substantially stationary state while being awake, and the like).

In this power saving control, the power consumption can be reduced by stopping the motion of at least one of the 3-axis acceleration sensor, the 3-axis gyro sensor, and the 3-axis geomagnetic sensor, and changing the sampling frequency of at least one of the 3-axis acceleration sensor, the 3-axis gyro sensor, and the 3-axis geomagnetic sensor to a predetermined sampling frequency. For example, in the power saving control, a combination of the activation or stop of each of the 3-axis acceleration sensor, the 3-axis gyro sensor, and the 3-axis geomagnetic sensor, and a combination of reduction of the sampling frequency by a predetermined ratio of each of the 3-axis acceleration sensor, the 3-axis gyro sensor, and the 3-axis geomagnetic sensor are set in advance as the condition for executing the power saving control. In a case where the estimation result of the behavior estimation unit 212 indicates the state in which the behavior of the monitored animal 6 is not active, the power saving control unit 508 performs the power saving control of each sensor on the basis of the preset condition for executing the power saving control. In addition, in a case where the estimation result indicates a state in which the behavior of the monitored animal 6 is active (For example, the state is a state other than the sleeping state and the resting state, and is referred to as an “active state”), the power saving control unit 508 stops the power saving control.

### (Method 2)

The power saving control unit 508 executes the power saving control of the motion detection unit 502 of the monitoring device 5 on the basis of the contents (that is, the power saving mode (power saving setting data)) set by the power saving mode setting unit 506 and stored in the power saving mode information storage unit 516. That is, the power saving control unit 508 executes the power saving control by controlling the activation or stop of each sensor of the 9-axis motion sensor according to the contents set by the power saving mode setting unit 506. Note that the power saving control unit 508 can individually control the activation or stop of each sensor of the 9-axis motion sensor, and can simultaneously control all the sensors to simultaneously activate or stop the sensors.

### (Method 3)

The power saving control unit 508 may execute the power saving control of the monitoring device 5 on the basis of the condition (power saving execution condition) output by the power saving setting estimation unit 233 of the server 2. Examples of the condition for executing the power saving control output by the power saving setting estimation unit 233 include conditions such as the activation and stop of each sensor of the 9-axis motion sensor and the reduction of the sampling frequency of each sensor. That is, the combination of the activation or stop of each of the 3-axis acceleration sensor, the 3-axis gyro sensor, and the 3-axis geomagnetic sensor, and the combination of the reduction of the sampling frequency by a predetermined ratio of each of the 3-axis acceleration sensor, the 3-axis gyro sensor, and the 3-axis geomagnetic sensor can be set as the condition for executing the power saving control. The power saving control unit 508 executes the power saving control by activating or stopping each sensor and reducing the sampling frequency of each sensor to a predetermined sampling frequency on the basis of the condition output by the power saving setting estimation unit 233.

### (Monitoring Device Storage Unit 510)

The monitoring device storage unit 510 stores an OS and various types of information regarding the animal monitoring system 1. The monitoring device storage unit 510 supplies predetermined information to a predetermined constituent member in response to a request from another constituent member. In addition, the monitoring device storage unit 510 stores information supplied from another constituent member in a predetermined storage unit.

### (Monitoring Device Storage Unit 510: Monitoring Device Information Storage Unit 512)

The monitoring device information storage unit 512 stores the information regarding the monitoring device 5 in association with the monitoring device identifier that identifies the monitoring device 5. The information regarding the monitoring device 5 is, for example, information such as the type of the monitoring device 5 and an installation location thereof.

### (Monitoring Device Storage Unit 510: Charge/Power Consumption Information Storage Unit 514)

The charge/power consumption information storage unit 514 stores the information detected by the power information detection unit 504. The charge/power consumption information storage unit 514 stores the information regarding the charging and discharging of the battery 524 and/or the information regarding the power consumption. The charge/power consumption information storage unit 514 stores these pieces of information in association with detection dates and times by the power information detection unit 504. In addition, the charge/power consumption information storage unit 514 may store histories of these pieces of information (that is, each history of the information regarding the charging and discharging and/or the information regarding the power consumption) for a certain period of time, and delete the histories after the certain period of time elapses.

### (Monitoring Device Storage Unit 510: Power Saving Mode Information Storage Unit 516)

The power saving mode information storage unit 516 stores the contents set by the power saving mode setting unit 506. That is, the power saving mode information storage unit 516 stores, as the power saving mode, a combination of information indicating the acceleration threshold and the like used to activate or stop the power saving control of each sensor constituting the motion detection unit 502. The power saving mode information storage unit 516 can store the information indicating the acceleration threshold that is an individual criterion for determination of the activation or stop of each sensor for the purpose of individually controlling the activation or stop of each sensor of the 9-axis motion sensor, and can store the information indicating the acceleration threshold that is a common criterion for determination of the activation or stop of all the sensors for the purpose of simultaneously controlling the activation or stop of all the sensors.

### (Monitoring Device Storage Unit 510: Power Saving Information Storage Unit 518)

The plurality of power saving modes (power saving setting data) can be set in advance as the power saving control of the monitoring device 5. The power saving mode has the contents defining in advance the execution of the activation, stop, or the like of each sensor of the 9-axis motion sensor, for example, on the basis of the acceleration value detected by the 3-axis acceleration sensor of the motion detection unit 502. That is, the plurality of power saving modes are configured by individually setting, for each sensor, which one of the 3-axis acceleration sensor, the 3-axis gyro sensor, and the 3-axis geomagnetic sensor is to be activated or stopped, a sampling frequency value of each sensor in the case of activation, and an acceleration value that is a determination criterion in the case of activation. As an example, in a case where the detected acceleration is x (m/s²), the power saving mode is set in which all of the 3-axis acceleration sensor, the 3-axis gyro sensor, and the 3-axis geomagnetic sensor are activated, and in a case where the acceleration is y (where x>y), the power saving mode is set in which the 3-axis acceleration sensor is activated and the 3-axis gyro sensor and the 3-axis geomagnetic sensor are stopped. In this manner, the plurality of power saving modes of the monitoring device 5 can be set in advance. The power saving information storage unit 518 stores the plurality of power saving modes. In addition, the power saving information storage unit 518 may store the power saving mode selected by the user via the information terminal 7, which is the power saving mode stored in the power saving mode information storage unit 516, as the power saving mode to be preferentially used.

### (Monitoring Device Communication Unit 520)

The monitoring device communication unit 520 communicates with the relay device 4 and the information terminal 7 so as to enable bidirectional communication by short-range wireless communication. In addition, the monitoring device communication unit 520 may directly communicate with the server 2 and/or the information terminal 7 via the communication network 3 (not via the relay device 4) so as to enable bidirectional communication. Note that each constituent member included in the monitoring device 5 acquires predetermined information from the server 2, the relay device 4, and/or the information terminal 7 via the monitoring device communication unit 520, and supplies predetermined information to a predetermined constituent member of the server 2, the relay device 4, and/or the information terminal 7 via the monitoring device communication unit 520.

### (Monitoring Device Output Unit 522)

The monitoring device output unit 522 simply outputs the motion status and the like of the monitoring device 5. The monitoring device output unit 522 is, for example, an indicator that can be configured by a light emitting diode (LED) or the like, and simply indicates a situation in which predetermined information is supplied from the monitoring device 5 to the server 2 or the like, a predetermined situation (charging and discharging situation) of the battery of the monitoring device 5, and/or a predetermined error, and the like by, for example, a light emission manner (e.g., a light emission color, the presence or absence of blinking, the interval of blinking, and the like).

As illustrated in FIG. 6(b), the information terminal 7 includes the input unit 700 that receives an input of predetermined information from the user, the output unit 702 that outputs predetermined information, an information terminal communication unit 704 that communicates with the server 2 and the relay device 4, and an information terminal storage unit 706 that stores predetermined information.

### (Input Unit 700, Output Unit 702)

The input unit 700 receives an input of a predetermined instruction, operation, and the like from the user. The input unit 700 supplies the instruction to a predetermined constituent member of the animal monitoring system 1. Each constituent member receiving the instruction exerts a predetermined function. The input unit 700 is an input device (e.g., a touch panel, a touch pad, a pointing device such as a mouse, a keyboard, a motion sensor, and the like) for receiving the operation input from the user.

The output unit 702 outputs various types of information regarding the execution of the animal monitoring system 1 (e.g., text information, image information of a still image or a moving image, audio information, and the like). The output unit 702 outputs various processing results and the information stored in the server storage unit 214, the relay device storage unit 408, the monitoring device storage unit 510, and/or the information terminal storage unit 706 in a user-perceptible manner. Specifically, the output unit 702 outputs various processing results in each constituent member and the information stored in each storage unit and the information terminal storage unit 706 as data in a predetermined format, a still image, a moving image, and/or a text. The output unit 702 may output information received from an external server. Note that the output unit 702 may include a display unit that displays various types of information, an audio output unit such as a speaker that outputs sound, and the like. The display unit may be, for example, a liquid crystal display or an organic EL display.

For example, the input unit 700 receives an instruction to acquire the rectal temperature and/or the behavior of the monitored animal 6 from the user. The rectal temperature estimation unit 210 and/or the behavior estimation unit 212, or the estimation result information storage unit 228 of the server 2 supplies the information regarding the rectal temperature and/or the estimated behavior to the output unit 702. The output unit 702 outputs the received information in a user-perceptible manner. Even when not receiving the acquisition instruction from the input unit 700, the rectal temperature estimation unit 210 and the behavior estimation unit 212 perform the estimation of the rectal temperature and the estimation of the behavior as needed, and continue to store the estimation results in the estimation result information storage unit 228. Therefore, the user can grasp not only the rectal temperature and/or the estimated behavior of the monitored animal 6 at a time point when giving the acquisition instruction via the information terminal 7, but also specify a time point earlier than the time point when giving the acquisition instruction and grasp the rectal temperature and/or the estimated behavior at the earlier time point.

### (Information Terminal Communication Unit 704)

The information terminal communication unit 704 communicates with the server 2 and the relay device 4 via the communication network 3 so as to enable bidirectional communication. In addition, the information terminal communication unit 704 can also communicate with the relay device 4 so as to enable bidirectional communication by short-range wireless communication. Note that each constituent member included in the information terminal 7 acquires predetermined information from the server 2, the relay device 4, and/or the monitoring device 5 via the information terminal communication unit 704, and supplies predetermined information to a predetermined constituent member of the server 2, the relay device 4, and/or the monitoring device 5 via the information terminal communication unit 704.

### (Information Terminal Storage Unit 706)

The information terminal storage unit 706 stores an OS and information regarding the information terminal 7. The information regarding the information terminal 7 is, for example, information regarding the user who uses the information terminal 7, a telephone number, a mail address, and/or other text information and image information.

Note that an application for the animal monitoring system 1 may be installed in the information terminal 7. This application can be configured to have the following functions. That is, the application can include a user authentication unit that performs user authentication, a behavior output unit that outputs, to the output unit 702, the behavior indicated by the estimated behavior information of the monitored animal 6 received from the server 2, a feature input unit that receives, via the input unit 700, an operation of registering the monitored animal 6 in the server 2 and an input of the feature amount of the monitored animal 6 to be supplied to the server 2, a rectal temperature output control unit that causes the output unit 702 to output the rectal temperature of the monitored animal 6 estimated in the server 2, a relay device output control unit that causes the output unit 702 to output the information regarding the relay device 4 linked with the information terminal 7, and an alarm unit that causes the output unit 702 to output the warning information from the warning unit 232 regarding the monitored animal 6 by push notification.

Here, each of the server 2, the relay device 4, the monitoring device 5, and the information terminal 7 is configured by connecting each constituent member of each of the server 2, the relay device 4, the monitoring device 5, and the information terminal 7 to a bus to which a general-purpose constituent element such as a central processing unit (CPU) and a RAM (not illustrated) is connected.

### [Processing Flow of Animal Monitoring System 1]

FIGS. 7 to 16 illustrate outline examples of processing flows in the animal monitoring system according to the present embodiment and outline examples of output contents in the information terminal. Note that the number of relay devices and the number of monitoring devices are not limited as long as the number is one or more. In addition, one relay device and a plurality of monitoring devices may be linked.

### (User Registration and Device Linking Processing)

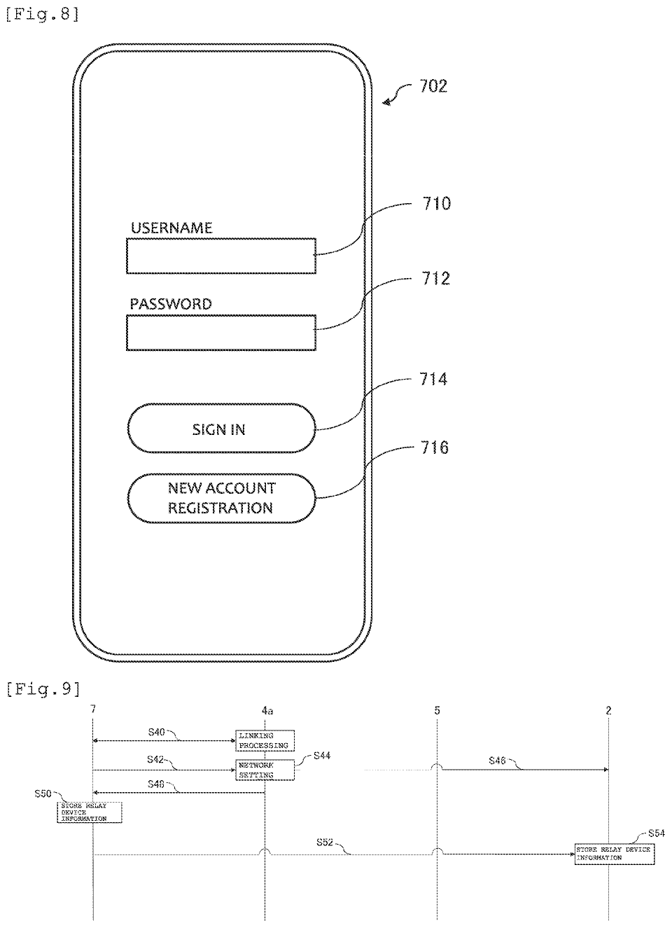

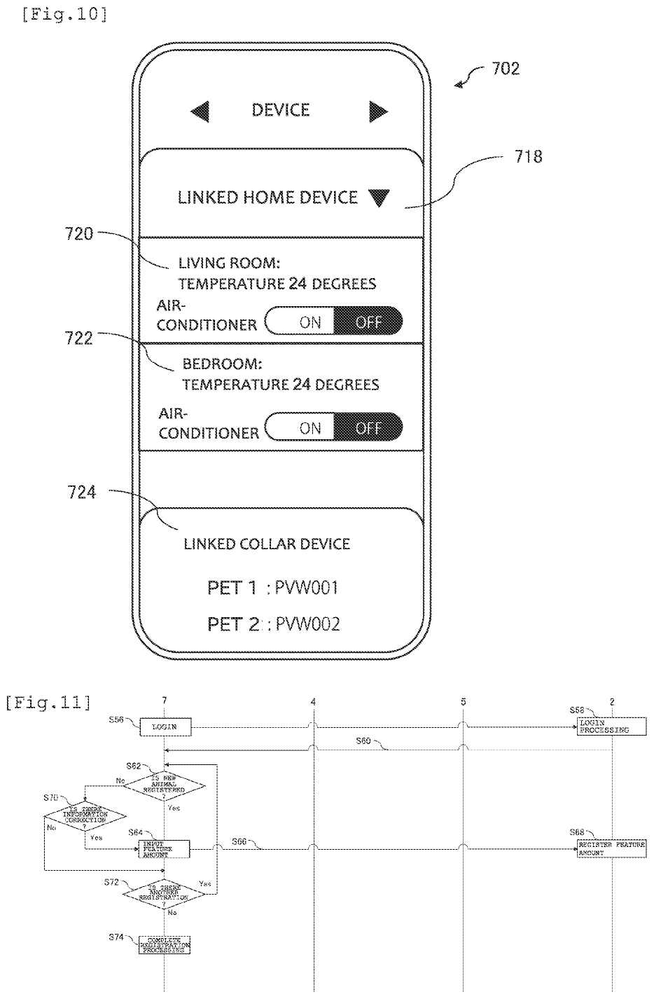

FIGS. 7 and 9 illustrate outline examples of a flow of user registration and device linking processing. In addition, FIG. 8 illustrates an outline example of output contents in the information terminal at the time of user registration, and FIG. 10 illustrates an outline example of output contents of information regarding the linked relay device in the information terminal.

First, when the relay device 4 is turned on, the relay device 4 and the monitoring device 5 are linked with each other to be initialized, and the relay device 4 and the server 2 are linked with each other. That is, as illustrated in FIG. 7, the linking control unit 400 connects the relay device 4 and the information terminal 7 and connects the relay device 4 and the monitoring device 5 by short-range wireless communication, and executes linking processing between the relay device 4 and the information terminal 7 (Step 10. Hereinafter, the step is referred to as “S”) and executes linking processing between the relay device 4 and the monitoring device 5 (S12) in response to a linking instruction received from the user via the information terminal 7.

In addition, the information terminal 7 is connected to the server 2 via the communication network 3. Here, the information terminal 7 performs local network setting of the relay device 4 in response to an instruction (S14) of network setting related to the relay device 4 from the user (S16), and connects the relay device 4 and the server 2 via the communication network 3 so as to enable bidirectional communication (S18). In the local network setting, a router may be provided between the relay device 4 and the communication network 3, and the local network setting may be performed on the router. The information terminal 7 acquires the information regarding the relay device 4 linked therewith from the relay device 4 (S20), and stores the acquired information regarding the relay device 4 in the information terminal storage unit 706 (S22).

In addition, the authentication registration unit 200 of the server 2 requests the information terminal 7 to register the user (S24). That is, the authentication registration unit 200 requests the information terminal 7 to create new account information of the user. For example, as illustrated in FIG. 8, the authentication registration unit 200 causes the output unit 702 of the information terminal 7 to display a username input field 710 to which the username is input, a password input field 712 to which the password is input, a sign-in icon 714 for requesting sign-in to the server 2, and a new account registration icon 716 for requesting new account registration. In response to an input instruction for the new account registration icon 716, the information terminal 7 supplies information about the new account registration to the authentication registration unit 200 (S26). The authentication registration unit 200 creates the account information of the user (the user ID and the password) using the information. As a result, the account of the user is issued (S28). Note that the authentication registration unit 200 stores the account information of the user in the user information storage unit 216.

In addition, the authentication registration unit 200 acquires the information regarding the relay device 4 stored in the information terminal storage unit 706 from the information terminal 7 (S30). The authentication registration unit 200 associates the user of the information terminal 7 with the information regarding the relay device 4 acquired from the information terminal 7, and stores and registers the information in the relay device information storage unit 220 (S32). That is, the authentication registration unit 200 stores the user identifier or the user account information in the relay device information storage unit 220 in association with the relay device identification information of the relay device 4.

On the other hand, the relay device 4 acquires the information regarding the monitoring device 5 linked therewith (for example, the monitoring device identification information or the like of the monitoring device 5) from the monitoring device 5 (S34), and supplies the information to the authentication registration unit 200 (S36). The authentication registration unit 200 associates the user of the information terminal 7 with the information regarding the monitoring device 5 acquired from the relay device 4, and stores and registers the information in the monitoring device information storage unit 222 (S38). That is, the authentication registration unit 200 stores the user identifier or the user account information in the monitoring device information storage unit 222 in association with the monitoring device identification information of the monitoring device 5.

In addition, as illustrated in FIG. 9, a relay device 4a different from the relay device 4 can also be linked with the server 2 and the information terminal 7. In this case, the authentication registration unit 200 receives the input of the username to the username input field 710, the input of the password to the password input field 712, and the input operation of the sign-in icon 714 by the user, which are output to the output unit 702 as illustrated in FIG. 8, and the user is allowed to log in to the animal monitoring system 1. The linking control unit 400 connects the relay device 4a and the information terminal 7 by short-range wireless communication, and executes linking processing between the relay device 4a and the information terminal 7 in response to a linking instruction received from the user via the information terminal 7 (S40).

In addition, the information terminal 7 is connected to the server 2 via the communication network 3, performs local network setting of the relay device 4a in response to an instruction (S42) of network setting related to the relay device 4a from the user (S44), and connects the relay device 4a and the server 2 via the communication network 3 so as to enable bidirectional communication (S46). The information terminal 7 acquires the information regarding the relay device 4a linked therewith from the relay device 4a (S48), and stores the acquired information regarding the relay device 4a in the information terminal storage unit 706 (S50). The authentication registration unit 200 acquires the information regarding the relay device 4a stored in the information terminal storage unit 706 from the information terminal 7 (S52), associates the user of the information terminal 7 with the information regarding the relay device 4a acquired from the information terminal 7, and stores and registers the information in the relay device information storage unit 220 (S54).

In this case, as illustrated in FIG. 10, the authentication registration unit 200 may also cause the output unit 702 of the information terminal 7 to display the plurality of relay devices so that the user can grasp the relay devices. That is, the authentication registration unit 200 causes the output unit 702 to output information regarding one or more relay devices linked with the server 2 in a user-perceptible manner. In the example of FIG. 10, the authentication registration unit 200 receives a user's instruction for a predetermined tab 718 (a tab displayed as “linked home device” in the example of FIG. 10), and outputs the information regarding the relay devices linked with the server 2. In the example of FIG. 10, one or more relay device display fields (that is, a relay device display field 720 and a relay device display field 722) are displayed in the tab 718, and each relay device display field displays a name set by the user for the relay device, a measurement result (for example, the temperature or the like) by the environment measurement unit 402 of the relay device, a motion status of an air conditioning system (air conditioner) when the air conditioning system is installed in an area where the relay device is installed, and the like. Note that the authentication registration unit 200 may also cause the output unit 702 to display information regarding one or more monitoring devices linked with the relay device (displayed as “linked collar device” in the example of FIG. 10) in a monitoring device display field 724.

### (Registration of Monitored Animal and Feature Amount)

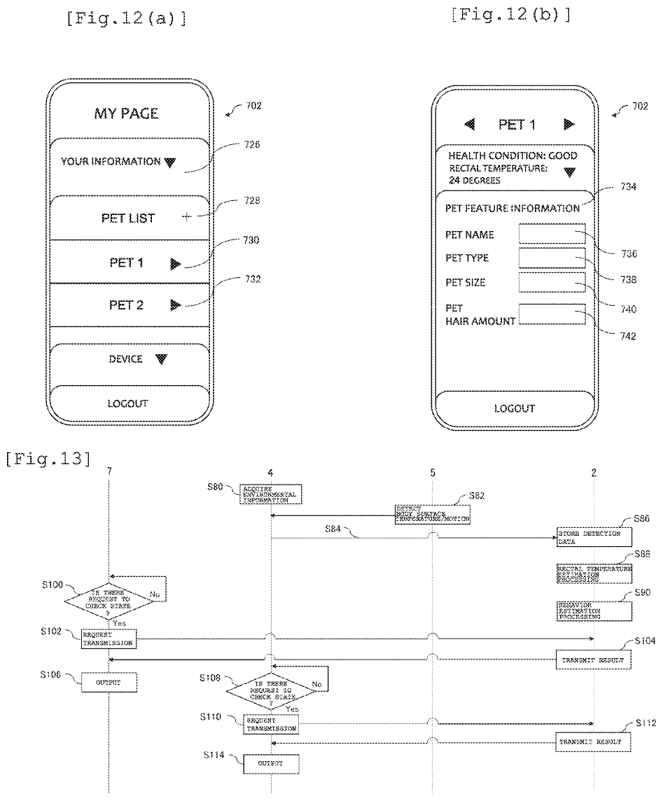

FIG. 11 illustrates an outline example of a processing flow when the monitored animal and the feature amount are registered in the server. Note that the monitored animal is, for example, the pet of the user (Hereinafter, the monitored animal may be referred to as a “pet”). In addition, FIG. 12 illustrates an example of output contents in the information terminal when the monitored animal is registered. That is, FIG. 12(a) illustrates an example in which a registered animal list is output to the information terminal when the monitored animal is registered, and FIG. 12(b) illustrates an example of an output screen of the information terminal when a new monitored animal is registered.

First, the user logs in to the animal monitoring system 1 from the information terminal 7 (S56). In a case where the account information of the user is stored in the user information storage unit 216, the authentication registration unit 200 continues login processing to complete the login, and in a case where the account information is not stored, the authentication registration unit 200 ends the login processing (S58). Next, the registration unit 202 requests the information terminal 7 to register the new monitored animal (S60). Note that the request may be executed by a feature amount input unit of a predetermined application in a case where the application is installed in the information terminal 7. In a case where the new monitored animal is to be registered by the user (Yes at S62), the input unit 700 of the information terminal 7 receives inputs of the information regarding the new monitored animal (for example, the name and the like of the monitored animal), and the feature amount of the new monitored animal (for example, the kind (type), size, hair amount, and the like of the animal) from the user (S64). The registration unit 202 acquires the input information regarding the new monitored animal, and the feature amount acquisition unit 204 acquires the input feature amount (S66). Subsequently, the registration unit 202 registers the new monitored animal in the server 2, and the feature amount acquisition unit 204 stores the feature amount of the monitored animal in the monitored animal information storage unit 218 in association with the monitored animal identification information of the monitored animal (S68). The feature amount stored in the monitored animal information storage unit 218 is used in the model generation unit 208 and the like. After S64, the registration unit 202 causes the output unit 702 to display a content inquiring of the user whether there is another new monitored animal to be further registered (S72). In a case where there is another new monitored animal or the feature amount of the already registered monitored animal is to be corrected (Yes at S72), the steps from S62 are repeated. On the other hand, in a case where there is no other new monitored animal and it is not necessary to correct the feature amount of the already registered monitored animal (No at S72), the registration unit 202 completes the registration processing (S74).

For example, as illustrated in FIG. 12(a), the registration unit 202 or the feature amount input unit of the application receives, via the input unit 700, an instruction from the user for a tab 726 where the information regarding the user is displayed. The registration unit 202 or the feature amount input unit displays, on the output unit 702, a list of information regarding the pets as the monitored animals owned by the user. The output unit 702 displays, for example, a button for displaying a page where the information regarding the pets already owned by the user is posted (for example, a monitored animal selection button 730 and a monitored animal selection button 732), and a new monitored animal registration button 728 for registering the new monitored animal. When receiving an input instruction for the monitored animal selection button 730 or the monitored animal selection button 732 via the input unit 700, the registration unit 202 refers to the monitored animal information storage unit 218 and causes the output unit 702 to output the information regarding the corresponding monitored animal. On the other hand, when receiving an input instruction for the new monitored animal registration button 728 via the input unit 700, the registration unit 202 or the feature amount input unit causes the output unit 702 to display a registration page 734 for registering the new monitored animal as illustrated in FIG. 12(b).

The registration page 734 includes an input area that receives the input of the information regarding the new monitored animal (for example, an input area 736 that receives the input of the name of the pet, an input area 738 that receives the input of the kind (type) of the pet, an input area 740 that receives the input of the size of the pet, an input area 742 that receives the input of the hair amount of the pet, and the like). For example, the registration unit 202 uses the pet name input to the input area 736 to execute the registration processing of the pet in the server 2. In addition, the feature amount acquisition unit 204 acquires the feature amount of the pet input to the input area 738 to the input area 742.

On the other hand, in a case where the user does not request the registration of the new monitored animal (No at S62), the registration unit 202 causes the output unit 702 to display a content inquiring of the user whether the feature amount of the already registered monitored animal is to be corrected (S70). In a case where there is a correction (Yes at S70), the registration unit 202 and the feature amount acquisition unit 204 execute steps similar to those from S64 except for the registration processing of the new monitored animal. In a case where there is no correction (No at S70), the steps from S72 are executed.

Through the above steps from S10 to S74, the predetermined information regarding each of the user, the monitored animal 6, the information terminal 7, the relay device 4, and the monitoring device 5 is stored and registered in the predetermined storage unit of the server 2.

### (Rectal Temperature Estimation Processing and Behavior Estimation Processing)

FIG. 13 illustrates an outline example of a flow of estimation processing of the rectal temperature and the behavior of the monitored animal.

The environment measurement unit 402 of the relay device 4 measures the environmental situation around the relay device 4 (S80). For example, the environment measurement unit 402 measures the temperature and/or the humidity as the environmental situation. In addition, the body surface temperature detection unit 500 of the monitoring device 5 detects the body surface temperature of the monitored animal, and supplies the information on the detected body surface temperature (body surface temperature information) to the relay device 4 (S82). Furthermore, the motion detection unit 502 of the monitoring device 5 detects the motion and/or the direction of the monitored animal and supplies the motion information (In a case where the motion detection unit 502 is configured by the 9-axis motion sensor, the information is the detection value by each sensor) about the detected motion and/or direction to the relay device 4 (S82).

The environment measurement unit 402 supplies the measurement result as the environmental information to the information acquisition unit 206 via the relay communication unit 418 (S84). In addition, the relay communication unit 418 supplies the body surface temperature information and/or the motion information received from the monitoring device 5 to the information acquisition unit 206 (S84). The information acquisition unit 206 stores the environmental information, the body surface temperature information, and the motion information in the detection information storage unit 226, and supplies these pieces of information to the rectal temperature estimation unit 210, the behavior estimation unit 212, and the warning unit 232 (S86). Here, the environment measurement unit 402, the body surface temperature detection unit 500, and the motion detection unit 502 continue to measure or detect the environmental information, the body surface temperature information, and the motion information, respectively, even when the information terminal 7 is not connected to the server 2 or when the predetermined application installed in the information terminal 7 is not activated. Even when the information terminal 7 is not connected to the server 2 or when the predetermined application installed in the information terminal 7 is not activated, the information acquisition unit 206 acquires the environmental information, the body surface temperature information, and the motion information at predetermined time intervals (for example, at intervals of one second), stores the information in the detection information storage unit 226, and supplies the information to the rectal temperature estimation unit 210, the behavior estimation unit 212, and the warning unit 232.

Subsequently, the rectal temperature estimation unit 210 estimates the rectal temperature of the monitored animal on the basis of at least the received body surface temperature information (S88). The rectal temperature estimation unit 210 may estimate the rectal temperature by further using the environmental information and/or the motion information. Specifically, the rectal temperature estimation unit 210 applies the body surface temperature indicated by the received body surface temperature information to the rectal temperature estimation model and outputs the estimated rectal temperature. The rectal temperature estimation unit 210 stores the output rectal temperature information in the estimation result information storage unit 228.

In addition, the behavior estimation unit 212 estimates the behavior of the monitored animal on the basis of at least the received motion information (S90). The behavior estimation unit 212 may estimate the behavior by further using the body surface temperature information and/or the environmental information. Specifically, the behavior estimation unit 212 applies the received motion information (for example, the detection value of each sensor constituting the motion detection unit 502) to the behavior estimation model and outputs the behavior information of the estimated behavior. Then, the behavior estimation unit 212 stores the output behavior information in the estimation result information storage unit 228.

The input unit 700 of the information terminal 7 waits until the user inputs an instruction to make a request to check the state of the monitored animal (No at S100). When the instruction is made (Yes at S100), the input unit 700 requests the server 2 to transmit information regarding the state of the monitored animal (S102). When receiving the request to check the state of the monitored animal via the input unit 700 of the information terminal 7, the server communication unit 234 of the server 2 supplies the estimated rectal temperature and/or the estimated behavior information stored in the estimation result information storage unit 228 to the information terminal 7 (S104). The output unit 702 of the information terminal 7 outputs the received estimated rectal temperature and/or estimated behavior information (S106).

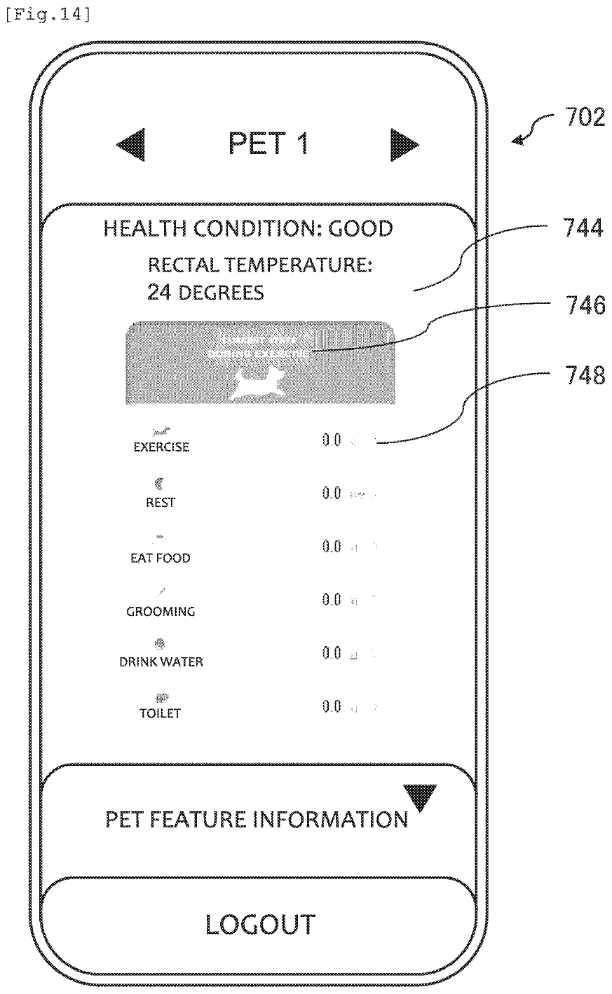

For example, as illustrated in FIG. 14, the output unit 702 can output a rectal temperature display area 744 that displays the received estimated rectal temperature, a behavior display area 746 that displays the behavior corresponding to the received estimated behavior information, and a history display area 748 that displays a cumulative time of each behavior taken by the monitored animal 6 so far for each behavior. In the example of FIG. 14, as an example, the rectal temperature display area 744 displays that the rectal temperature of the pet is “24 degrees”, and the behavior display area 746 displays that the pet's behavior is “during exercise”. In addition, the output unit 702 refers to the estimation result information storage unit 228, and displays, in the history display area 748, for example, a time when the pet takes each behavior of “exercise”, “rest”, “eat food”, “grooming”, “drink water”, and “toilet”, and the cumulative time and the cumulative number of times of each behavior.

In addition, the relay device 4 may include a relay device input unit that receives an input of a predetermined instruction by the user. The relay device input unit waits until the user inputs an instruction to make a request to check the state of the monitored animal (No at S108). When the instruction is made (Yes at S108), the relay device input unit requests the server 2 to transmit information regarding the state of the monitored animal (S110). When receiving the request to check the state of the monitored animal via the relay device input unit, the server communication unit 234 of the server 2 supplies the estimated rectal temperature and/or the estimated behavior information stored in the estimation result information storage unit 228 to the relay device 4 (S112). The output unit 420 of the relay device 4 outputs the received estimated rectal temperature and/or estimated behavior information similarly to the above description (S114).

### Example 1 of Power Saving Control Processing

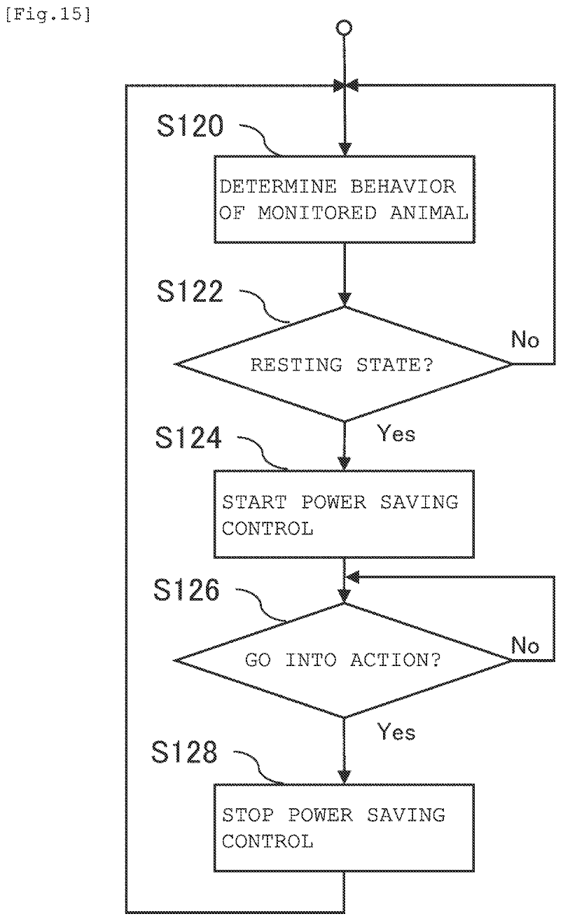

FIG. 15 illustrates an outline example of a processing flow of the power saving control in the monitoring device.

First, the behavior estimation unit 212 of the server 2 determines the behavior of the monitored animal 6 on the basis of the motion information of the monitored animal 6 acquired from the monitoring device 5 and the behavior estimation model (S120). The motion information in this case is the detection value of each sensor in a case where all the sensors included in the 9-axis motion sensor as the motion detection unit 502 are operating. In a case where the result of determining the behavior by the behavior estimation unit 212 indicates that the monitored animal 6 is not in a resting state (No at S122), the power saving setting unit 406 of the relay device 4 stands by without executing the power saving control.

On the other hand, in a case where the result of determining the behavior by the behavior estimation unit 212 indicates that the monitored animal 6 is in a resting state (Yes at S122), the power saving setting estimation unit 233 outputs the condition (power saving execution condition) for causing the monitoring device 5 to execute the power saving control on the basis of the motion information of the monitored animal 6 in the resting state and the power saving setting estimation model. The power saving execution condition may be, for example, a condition for stopping the operation of a predetermined sensor of the 9-axis motion sensor and/or a condition for reducing the sampling frequency of a predetermined sensor to a predetermined frequency. Note that the power saving setting estimation unit 233 may set the power saving execution condition in advance without using the power saving setting estimation model. The power saving setting estimation unit 233 supplies the power saving execution condition to the power saving setting unit 406 of the relay device 4. The power saving setting unit 406 executes the power saving control of the monitoring device 5 on the basis of the power saving execution condition (S124). That is, the power saving control unit 508 of the monitoring device 5 is controlled by the power saving setting unit 406 to execute the power saving control by stopping the operation of some sensors of the 9-axis motion sensor, reducing the sampling frequency of some sensors of the 9-axis motion sensor to a predetermined frequency, and/or activating some sensors of the 9-axis motion sensor at regular time intervals to confirm whether the resting state continues (polling).

In a case where the result of determining the behavior of the monitored animal 6 by the behavior estimation unit 212 on the basis of the motion information of the monitored animal 6 acquired from the monitoring device 5 and the behavior estimation model indicates that the monitored animal 6 is in the resting state without going into action (No at S126), the power saving setting unit 406 of the relay device 4 stands by while executing the power saving control. On the other hand, in a case where the result of determining the behavior by the behavior estimation unit 212 indicates that the monitored animal 6 goes into action (Yes at S126), the power saving setting unit 406 stops the power saving control (S128). As a result, the operations of all the sensors included in the 9-axis motion sensor as the motion detection unit 502 are started again. Thereafter, the steps from S120 are repeated.

### Example 2 of Power Saving Control Processing

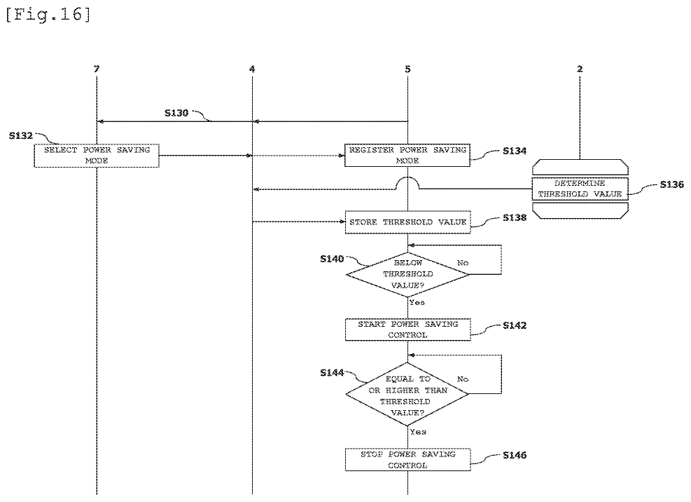

FIG. 16 illustrates another outline example of the processing flow of the power saving control in the monitoring device.

The power saving control of the monitoring device 5 may also be executed on the basis of the behavior information of the monitored animal 6 estimated by the server 2, and the information regarding the charging of the monitoring device 5 and/or the information regarding the power consumption of the monitoring device 5. For example, the power saving control can be performed by appropriately setting the power saving mode including a predetermined threshold for switching the execution/stop of the power saving control on the motion detection unit 502 by the power saving control unit 508 on the basis of the behavior information of the monitored animal 6.

First, the user refers to the one or more power saving modes stored in the power saving mode information storage unit 516 of the monitoring device 5 using the information terminal 7 via the relay device 4 (S130), and selects one power saving mode (S132). The information terminal 7 stores and registers the power saving mode selected by the user in the power saving information storage unit 518 as the power saving mode to be preferentially used (S134). Note that the one or more power saving modes stored in the power saving mode information storage unit 516 are not particularly limited, but the contents of the power saving modes may be set in advance as shown in Table 1 below.

TABLE 1

| | 3-axis | 3-axis | 3-axis | Body surface |
|---|---|---|---|---|
| | acceleration | gyro | geomagnetic | temperature |
| | sensor | sensor | sensor | detection unit |
| Power saving | Operate | Operate | Operate | Operate |
| mode OFF | | | | |
| Power saving | Operate | Operate | Stop | Operate |
| mode 1 | | | | |
| Power saving | Operate | Stop | Stop | Operate |
| mode 2 | | | | |
| Power saving | Stop | Stop | Stop | Stop |
| mode 3 | | | | |

That is, each power saving mode includes contents in which the operation or stop of each of the three sensors included in the 9-axis motion sensor as the motion detection unit 502 and the operation or stop of the body surface temperature detection unit 500 are set. The example of Table 1 illustrates a mode (power saving mode OFF) in which all of the sensors and the body surface temperature detection unit 500 are operated, a mode (power saving mode 1) in which only the 3-axis geomagnetic sensor is stopped, a mode (power saving mode 2) in which the 3-axis gyro sensor and the 3-axis geomagnetic sensor are stopped, and a mode (power saving mode 3) in which all of the sensors and the body surface temperature detection unit 500 are stopped. The combination of the operation or stop of the sensors and the body surface temperature detection unit 500 is not limited to Table 1, and may be another combination.

Subsequently, the power saving setting estimation unit 233 of the server 2 outputs a threshold for stopping the operation of each sensor of the 9-axis motion sensor as the motion detection unit 502 and/or a threshold for stopping the operation of the body surface temperature detection unit 500, as the condition for causing the monitoring device 5 to execute the power saving control, by using the body surface temperature and/or the motion of the animal acquired by the monitoring device 5, the information regarding the charging of the monitoring device 5, and/or the information regarding the power consumption of the monitoring device 5, and the power saving setting estimation model (S136). An acceleration value detected by the motion detection unit 502, which is a parameter that enables the most effective power saving control, is determined as the threshold (Note that the threshold may be determined for the detection value of angular velocity or geomagnetism instead of acceleration) on the basis of the power saving setting estimation model. Specifically, the power saving setting estimation unit 233 applies the motion information indicating the motion of the monitored animal 6, the information regarding the charging of the monitoring device 5, and/or the information regarding the power consumption in the monitoring device 5 to the power saving setting estimation model, and outputs an optimal threshold. Note that the threshold is calculated at predetermined time intervals. The power saving setting estimation unit 233 supplies the output threshold to the power saving mode information storage unit 516 of the monitoring device 5 via the relay device 4 (S138). In a modification, the user may manually set the threshold.

As a result, the power saving control unit 508 of the monitoring device 5 can execute the power saving control by reference to the acceleration threshold stored in the power saving mode information storage unit 516 and the registered power saving mode. That is, the power saving control unit 508 executes the power saving control according to the registered power saving mode (S142) when the acceleration detected by the motion detection unit 502 falls below the threshold stored in the power saving mode information storage unit 516 (Yes at S140), and stops the power saving control (S146) when the acceleration is equal to or higher than the threshold (Yes at S144). As a result, the power saving control of the monitoring device 5 is executed. When the acceleration detected by the motion detection unit 502 does not fall below the threshold stored in the power saving mode information storage unit 516 (No at S140), the power saving control is not executed. When the acceleration does not become equal to or higher than the threshold after the start of the power saving control (No at S144), the power saving control is continued.

### [Animal Monitoring Program]

Each constituent element included in the animal monitoring system 1 according to the present embodiment illustrated in FIGS. 1 to 16 can be achieved by causing an arithmetic processing device such as a central processing unit (CPU) to execute a program (that is, an animal monitoring program), that is, by processing by software. In addition, each constituent element can also be achieved by writing the program in advance to hardware as an electronic component such as an integrated circuit (IC). Note that software and hardware can be used in combination as well.

The animal monitoring program according to the present embodiment can be incorporated in advance in, for example, an IC, a ROM, or the like. In addition, the animal monitoring program can also be provided as a computer program by being recorded in a computer-readable recording medium such as a magnetic recording medium, an optical recording medium, or a semiconductor recording medium as a file in an installable format or an executable format. The recording medium storing the program may be a non-transitory recording medium such as a CD-ROM or a DVD. Furthermore, the animal monitoring program can be stored in advance in a computer connected to a communication network such as the Internet, and provided by being downloaded via the communication network.

The animal monitoring program according to the present embodiment causes the CPU and the like to allow the animal monitoring program to function as the authentication registration unit 200, the registration unit 202, the feature amount acquisition unit 204, the information acquisition unit 206, the model generation unit 208, the rectal temperature estimation unit 210, the behavior estimation unit 212, the server storage unit 214, the warning unit 232, the power saving setting estimation unit 233, the server communication unit 234, the linking control unit 400, the environment measurement unit 402, the position information detection unit 404, the power saving setting unit 406, the relay device storage unit 408, the charge control unit 414, the program update unit 416, the relay communication unit 418, the output unit 420, the body surface temperature detection unit 500, the motion detection unit 502, the power information detection unit 504, the power saving mode setting unit 506, the power saving control unit 508, the monitoring device storage unit 510, the monitoring device communication unit 520, the monitoring device output unit 522, the input unit 700, the output unit 702, the information terminal communication unit 704, and the information terminal storage unit 706 described in FIGS. 1 to 16.

### Effects of Embodiment

According to the animal monitoring system 1 according to the present embodiment, since the rectal temperature estimation model generated using the body surface temperatures and the rectal temperatures of a large number of monitored animals is used and the rectal temperature estimation model can be corrected using the feature amount of the actual monitored animal, the rectal temperature of the monitored animal of the user can be accurately estimated from the body surface temperature of the monitored animal. In addition, in the animal monitoring system 1, since the body surface temperature can be acquired without using an infrared ray sensor, the configuration of the monitoring device 5 can be simplified and miniaturized as compared with a conventional monitoring system, and thus can reduce the annoying feeling the monitored animal feels.

In addition, according to the animal monitoring system 1, the power saving control of the monitoring device 5 can be executed on the basis of the behavior estimated from the motion information of the monitored animal. Therefore, even in a case where the state of the monitored animal 6 is detected on a high level and with high accuracy using the body surface temperature detection unit 500 and the motion detection unit 502 (for example, the detection unit using the 9-axis motion sensor), it is possible to save the power consumption of the monitoring device 5, and reduce the charging frequency of the battery 524 of the monitoring device 5.

Note that the animal monitoring system, the animal monitoring server, the animal monitoring method, the animal monitoring program, and the learning model according to the present embodiment can also be mentioned in the following notes that should not be confused with the claims.

### (Note 1)

A pet monitoring system including a body surface temperature detection device that is attached to a pet to measure a body surface temperature of the pet, and a body surface temperature calculation unit that is connected to the temperature detection device, in which the body surface temperature detection device is configured to be attached to any position on a body surface of the pet excluding an anal portion, and the body surface temperature calculation unit includes: a pet feature value input unit that causes a computer to allow a user to input a feature value of the pet; a rectal temperature estimation model storage unit that causes the computer to store a rectal temperature estimation model defining a relationship between the body surface temperature of the pet and a rectal temperature of the pet according to the feature of the pet; and a rectal temperature estimation calculation unit that causes the computer to calculate the rectal temperature of the pet by applying the body surface temperature of the pet detected by the body surface temperature detection device and the feature of the animal to the rectal temperature estimation model, and output the rectal temperature such that the rectal temperature can be displayed to the user.

### (Note 2)

An animal monitoring server capable of estimating a rectal temperature of a monitored animal that is a companion animal, a rearing animal, or livestock, the animal monitoring server including a rectal temperature estimation unit configured to estimate the rectal temperature of the monitored animal by applying a body surface temperature detected by the body surface temperature detection unit that is attached to the monitored animal and detects the body surface temperature of the monitored animal, to a rectal temperature estimation model that receives, as rectal temperature training data, a combination of the body surface temperature of the monitored animal and a feature amount including information regarding a hair amount at a position of the monitored animal where the body surface temperature detection unit is attached, and the rectal temperature of the monitored animal, and outputs the rectal temperature of the monitored animal from the body surface temperature and the feature amount of the monitored animal by machine learning using the rectal temperature training data.

### (Note 3)

An animal monitoring method in an animal monitoring system capable of estimating a rectal temperature of a monitored animal that is a companion animal, a rearing animal, or livestock, the animal monitoring method including
a rectal temperature estimation step of estimating the rectal temperature of the monitored animal by applying a body surface temperature detected by the body surface temperature detection unit that is attached to the monitored animal and detects the body surface temperature of the monitored animal, to a rectal temperature estimation model that receives, as rectal temperature training data, a combination of the body surface temperature of the monitored animal and a feature amount including information regarding a hair amount at a position of the monitored animal where the body surface temperature detection unit is attached, and the rectal temperature of the monitored animal, and outputs the rectal temperature of the monitored animal from the body surface temperature and the feature amount of the monitored animal by machine learning using the rectal temperature training data.

### (Note 4)

An animal monitoring program for an animal monitoring system capable of estimating a rectal temperature of a monitored animal that is a companion animal, a rearing animal, or livestock, the animal monitoring program causing a computer to achieve
a rectal temperature estimation function to estimate the rectal temperature of the monitored animal by applying a body surface temperature detected by the body surface temperature detection unit that is attached to the monitored animal and detects the body surface temperature of the monitored animal, to a rectal temperature estimation model that receives, as rectal temperature training data, a combination of the body surface temperature of the monitored animal and a feature amount including information regarding a hair amount at a position of the monitored animal where the body surface temperature detection unit is attached, and the rectal temperature of the monitored animal, and outputs the rectal temperature of the monitored animal from the body surface temperature and the feature amount of the monitored animal by machine learning using the rectal temperature training data.

### (Note 5)

A rectal temperature estimation model that causes a processor to function to output a rectal temperature of a monitored animal that is a companion animal, a rearing animal, or livestock when a body surface temperature of the monitored animal is input,
in which the rectal temperature estimation model is learned by using, as training data, a combination of the body surface temperature of the monitored animal and a feature amount including information regarding a hair amount at a position of the monitored animal where the body surface temperature detection unit is attached, and the rectal temperature of the monitored animal, and in the learning, a relationship of the rectal temperature with the body surface temperature and the feature amount is learned using the training data to estimate the rectal temperature of the monitored animal.

### REFERENCE SIGNS LIST

1 Animal monitoring system
2 Server
3 Communication network
4, 4a Relay device
5 Monitoring device
6 Monitored animal
7 Information terminal
10 Belt
11 Device mounting portion
31 Hole portion
40 Main body portion
42 Mounting portion
44 Insertion plug
200 Authentication registration unit
202 Registration unit
204 Feature amount acquisition unit
206 Information acquisition unit
208 Model generation unit
210 Rectal temperature estimation unit
212 Behavior estimation unit
214 Server storage unit
216 User information storage unit
218 Monitored animal information storage unit
220 Relay device information storage unit
222 Monitoring device information storage unit
224 Model information storage unit
226 Detection information storage unit
228 Estimation result information storage unit
230 Warning information storage unit
232 Warning unit
233 Power saving setting estimation unit
234 Server communication unit
400 Linking control unit
402 Environment measurement unit
404 Position information detection unit
406 Power saving setting unit
408 Relay device storage unit
410 Linking information storage unit
412 Power saving information storage unit
414 Charge control unit
416 Program update unit
418 Relay communication unit
420 Output unit
500 Body surface temperature detection unit
502 Motion detection unit
504 Power information detection unit
506 Power saving mode setting unit
508 Power saving control unit
510 Monitoring device storage unit
512 Monitoring device information storage unit
514 Charge/power consumption information storage unit
516 Power saving mode information storage unit
518 Power saving information storage unit
520 Monitoring device communication unit
522 Monitoring device output unit
524 Battery
700 Input unit
702 Output unit
704 Information terminal communication unit
706 Information terminal storage unit
710 Username input field
712 Password input field
714 Sign-in icon
716 New account registration icon
718, 726 Tab
720, 722 Relay device display field
724 Monitoring device display field
728 New monitored animal registration button
730, 732 Monitored animal selection button
734 Registration page
736, 738, 740, 742 Input area
744 Rectal temperature display area
746 Behavior display area
748 History display area

1. An animal monitoring system capable of estimating a rectal temperature of a monitored animal that is a companion animal, a rearing animal, or livestock, the animal monitoring system comprising:
a body surface temperature detection unit attached around a neck of the monitored animal and configured to detect a body surface temperature of the monitored animal; and
a rectal temperature estimation unit configured to estimate the rectal temperature of the monitored animal by applying a feature amount including information regarding a hair amount at a position of the monitored animal where the body surface temperature detection unit is attached, the body surface temperature detected at a predetermined time interval by the body surface temperature detection unit, and environmental information around the monitored animal at a position separated from the monitored animal in a space where the monitored animal is located, to a rectal temperature estimation model that receives, as rectal temperature training data, a combination of the body surface temperature of the monitored animal, the feature amount of the monitored animal, and the environmental information around the monitored animal, and the rectal temperature of the monitored animal, and outputs the rectal temperature of the monitored animal from the feature amount and the body surface temperature of the monitored animal, and the environmental information around the monitored animal by machine learning using the rectal temperature training data.

2. The animal monitoring system according to claim 1, wherein the rectal temperature received by the rectal temperature training data is the rectal temperature of the monitored animal when the body surface temperature of the monitored animal is detected.

3. The animal monitoring system according to claim 1 or 2, further comprising
a feature amount acquisition unit configured to acquire the feature amount of the monitored animal,
wherein the rectal temperature estimation unit estimates the rectal temperature by applying the body surface temperature, the feature amount acquired by the feature amount acquisition unit, and the environmental information to the rectal temperature estimation model.

4. The animal monitoring system according to claim 3, wherein the feature amount further includes at least one piece of information selected from a group consisting of information regarding a kind of the monitored animal and information regarding a size of the monitored animal.

5. The animal monitoring system according to claim 4, further comprising
an information acquisition unit configured to acquire the environmental information,
wherein the rectal temperature estimation unit estimates the rectal temperature by applying the feature amount, the body surface temperature, and the surrounding environmental information acquired by the information acquisition unit to the rectal temperature estimation model.

6. The animal monitoring system according to claim 1, further comprising:
a motion detection unit attached to the monitored animal and configured to detect a motion of the monitored animal; and
a behavior estimation unit configured to estimate a behavior of the monitored animal by applying motion information indicating the motion detected by the motion detection unit to a behavior estimation model that receives, as behavior training data, a combination of the motion information indicating the motion of the monitored animal and behavior information indicating the behavior of the monitored animal corresponding to the motion, and outputs the behavior information indicating the behavior of the monitored animal from the motion information of the monitored animal by machine learning using the behavior training data.

7. The animal monitoring system according to claim 1, further comprising
a warning unit configured to output warning information in a case where the rectal temperature estimated by the rectal temperature estimation unit is a temperature equal to or higher than a predetermined threshold.

8. The animal monitoring system according to claim 5, wherein the information acquisition unit acquires the environmental information from an environment measurement unit that is installed at the position separated from the monitored animal in the space where the monitored animal is located.

9. An animal monitoring server capable of estimating a rectal temperature of a monitored animal that is a companion animal, a rearing animal, or livestock, the animal monitoring server comprising
a rectal temperature estimation unit configured to estimate the rectal temperature of the monitored animal by applying a feature amount including information regarding a hair amount of the monitored animal at a position where a body surface temperature detection unit that is attached to the monitored animal and detects a body surface temperature of the monitored animal is attached, the body surface temperature detected at a predetermined time interval by the body surface temperature detection unit, and environmental information around the monitored animal at a position separated from the monitored animal in a space where the monitored animal is located, to a rectal temperature estimation model that receives, as rectal temperature training data, a combination of the body surface temperature around a neck of the monitored animal, the feature amount of the monitored animal, and the environmental information around the monitored animal, and the rectal temperature of the monitored animal, and outputs the rectal temperature of the monitored animal from the feature amount and the body surface temperature of the monitored animal, and the environmental information around the monitored animal by machine learning using the rectal temperature training data.

10. An animal monitoring method in an animal monitoring system capable of estimating a rectal temperature of a monitored animal that is a companion animal, a rearing animal, or livestock, the animal monitoring method comprising
a rectal temperature estimation step of estimating the rectal temperature of the monitored animal by applying a feature amount including information regarding a hair amount of the monitored animal at a position where a body surface temperature detection unit that is attached to the monitored animal and detects a body surface temperature of the monitored animal is attached, the body surface temperature detected at a predetermined time interval by the body surface temperature detection unit, and environmental information around the monitored animal at a position separated from the monitored animal in a space where the monitored animal is located, to a rectal temperature estimation model that receives, as rectal temperature training data, a combination of the body surface temperature around a neck of the monitored animal, the feature amount of the monitored animal, and the environmental information around the monitored animal, and the rectal temperature of the monitored animal, and outputs the rectal temperature of the monitored animal from the feature amount and the body surface temperature of the monitored animal, and the environmental information around the monitored animal by machine learning using the rectal temperature training data.

11. An animal monitoring program for an animal monitoring system capable of estimating a rectal temperature of a monitored animal that is a companion animal, a rearing animal, or livestock, the animal monitoring program causing a computer to achieve
a rectal temperature estimation function to estimate the rectal temperature of the monitored animal by applying a feature amount including information regarding a hair amount of the monitored animal at a position where a body surface temperature detection unit that is attached to the monitored animal and detects a body surface temperature of the monitored animal is attached, the body surface temperature detected at a predetermined time interval by the body surface temperature detection unit, and environmental information around the monitored animal at a position separated from the monitored animal in a space where the monitored animal is located, to a rectal temperature estimation model that receives, as rectal temperature training data, a combination of the body surface temperature around a neck of the monitored animal, the feature amount of the monitored animal, and the environmental information around the monitored animal, and the rectal temperature of the monitored animal, and outputs the rectal temperature of the monitored animal from the feature amount and the body surface temperature of the monitored animal, and the environmental information around the monitored animal by machine learning using the rectal temperature training data.

12. A rectal temperature estimation model that causes a processor to function to output a rectal temperature of a monitored animal that is a companion animal, a rearing animal, or livestock when a body surface temperature and information regarding a hair amount of the monitored animal and environmental information around the monitored animal are input,
wherein the rectal temperature estimation model is learned by using, as training data, a combination of the body surface temperature around a neck of the monitored animal, a feature amount including the information regarding the hair amount of the monitored animal at a position where a body surface temperature detection unit that is attached to the monitored animal and detects the body surface temperature of the monitored animal is attached, and the environmental information around the monitored animal at a position separated from the monitored animal in a space where the monitored animal is located, and the rectal temperature of the monitored animal, and
in the learning, a relationship of the rectal temperature with the body surface temperature, the feature amount, and the environmental information is learned using the training data to estimate the rectal temperature of the monitored animal.

13. An animal monitoring system capable of estimating a rectal temperature of a monitored animal that is a companion animal, a rearing animal, or livestock, the animal monitoring system comprising:
a body surface temperature detection unit attached to the monitored animal and configured to detect a body surface temperature of the monitored animal; and
a rectal temperature estimation unit configured to estimate the rectal temperature of the monitored animal by applying the body surface temperature detected by the body surface temperature detection unit to a rectal temperature estimation model that receives, as rectal temperature training data, a combination of the body surface temperature of the monitored animal and a feature amount including information regarding a hair amount at a position of the monitored animal where the body surface temperature detection unit is attached, and the rectal temperature of the monitored animal, and outputs the rectal temperature of the monitored animal from the body surface temperature and the feature amount of the monitored animal by machine learning using the rectal temperature training data.
